(19) United States
(12) Patent Application Publication
(10) Pub. No.: US 2026/0185167 A1
(43) Pub. Date: Jul. 2, 2026

(54) METHODS FOR DETECTING AND QUANTIFYING THE PRESENCE OF AN ORGANISM IN A SAMPLE

(71) Applicant: SYSTEMS BIOLOGY LABORATORY UK, ABINGDON OXFORDSHIRE (GB)

(72) Inventors: Nick Parkinson, Abingdon Oxfordshire (GB); Mike Fischer, Abingdon Oxfordshire (GB)

(73) Assignee: SYSTEMS BIOLOGY LABORATORY UK, ABINGDON OXFORDSHIRE (GB)

(21) Appl. No.: 19/113,147

(22) PCT Filed: Sep. 20, 2023
(86) PCT No.: PCT/GB2023/052433

§ 371 Date: Mar. 19, 2025

(30) Foreign Application Priority Data
Sep. 20, 2022 (GB) 2213734.3

(51) Int. Cl. C12Q 1/689; C12Q 1/6869; C12Q 1/6895

(52) U.S. Cl. CPC C12Q 1/689; C12Q 1/6869; C12Q 1/6895; C12Q 2600/156

(57) ABSTRACT

The invention relates to methods for detecting and quantifying the presence of an organism in a sample, typically for detecting and quantifying the presence of a non-host organism in a host, and kits providing components useful for performing such methods. The invention also relates to methods for monitoring the effectiveness of a treatment of a disease or infection associated with a pathogen in a host. The invention further relates to methods for treating a disease or an infection associated with a pathogen in a host. The invention additionally relates to a computer program and to a computer-readable storage medium, comprising instructions causing a computer to carry out the method of the invention.

### FIELD OF THE INVENTION

The present invention relates to a method for detecting and quantifying the presence of an organism in an environment, typically for detecting and quantifying the presence of a non-host organism in a host.

### BACKGROUND OF THE INVENTION

Traditional methods for detecting the presence of an organism are often time consuming and work intensive. For example, many of these methods require the step of culturing the organism in a sterile state in order to prevent contamination from an initial sample. Once the culture is established, the method typically relies on extraction and then amplification and/or sequencing of deoxyribonucleic acids (DNA) or ribonucleic acids (RNA).

Current techniques in the art for identifying an organism, particularly microorganisms, also offer limited genetic resolution to differentiate between closely related species. These techniques also require time consuming and extensive experimental protocols. Furthermore, these techniques often only identify species that have been preselected for investigation and therefore provide a limited view on the organisms present and do not provide quantitation of the organisms. In particular, there is a need to provide improved methods for both detecting and quantifying the presence of an organism in an environment (such as a host sample). There is also a need for methods which offer high levels of genetic resolution, reduced experimental burden and/or an increased range of organisms to be detected and quantitated.

The ability to accurately quantify the amount of a non-host organism present in a sample obtained from a host is critical information in combination with the non-host organisms present. The presence alone of a non-host organism is not indicative that such an organism is the pathogen responsible for an infection. Multiple non-host organisms exist within a host in mutualistic or commensal relationships when present at certain amounts. However, if these organisms become present at excessive amounts the relationship can become pathogenic. In addition, multiple non-host organisms with the potential for causing an infection can be present in a host at one time and therefore determining which non-host organism or combination of non-host organisms is/are responsible for the infection requires accurate information on the amount of each non-host organism. It is therefore advantageous to have a method that can provide absolute quantities of the organisms present in a sample, such as by cell number.

The ability to reliably and accurately detect the presence of foreign organism in an environment has multiple applications including identification and quantification of a pathogen in the clinical setting such as diagnosing an infection, identification of an organism contaminating an environment such as identifying an alien species, identification of an organism responsible for an outbreak such as the source of food poisoning and cataloguing the presence of different organisms in specific environments.

There is in particular a need in a clinical setting to reliably and accurately identify and quantify non-host organisms in a sample obtained from a host in a time efficient manner. The time it takes to correctly identify the pathogen responsible for an infection can have direct consequences on patient treatment options and the patient outcome. If an infection is diagnosed but the wrong pathogen is identified as being responsible for the infection, this can result in implementing the incorrect treatment plan resulting in unnecessary/ineffective medication administered to the patient. This increases the period of recovery for the patient and requires a lack of progress in the treatment plan to be identified and subsequent/further tests conducted to allow correct identification of the pathogen and identification of appropriate treatment options. Identification of appropriate treatment options is further complicated by the continuing emergence of antibiotic resistant strains. The continuing emergence of antibiotic resistant strains limits the treatment options for patients and creates a further need to administer the appropriate therapy for an infection to reduce the likelihood of unsuccessfully treating an infection and limit introducing unnecessary antibiotics into the environment. Delays in successful treatment of patients due to incorrect identification of the pathogen not only impact the patients' health and quality of life but also increase the burden on health care providers requiring increased medical attention, further diagnostic tests and administration of additional therapies.

### SUMMARY OF INVENTION

The inventors have developed a means of accurately detecting and quantifying an organism in an environment, to address the limitations of previous methods used to detect organisms, particularly non-host organisms in samples obtained from a host. The inventors conceived that quantification of an amount of an organism in a sample based on the amount of a sequenced organism nucleic acid sequence would provide an accurate and direct determination of the relevance of the organism in the sample, for various applications such as detecting and quantifying a non-host organism in a sample obtained from the host. The methods of the invention also allow for directly obtaining and sequencing non-host organism nucleic acid from the sample without amplification and without contamination by host cell DNA when the sample is obtained from a host. The examples illustrate the improved efficiency and accuracy of detection and quantitation of non-host organisms compared to previous methods.

The invention provides a method for detecting and quantifying the presence of an organism in a sample. In preferred embodiments of all aspects described below unless stated to the contrary, the organism is a non-host organism, the reference organism is a non-host organism and the sample is obtained from a host. The method comprises determining an amount of sequenced organism nucleic acid sequence in the sample using sequencing data comprising at least one sequenced organism nucleic acid sequence obtained from sequencing organism nucleic acid from the sample. Obtaining the sequencing data may comprise obtaining organism nucleic acids from a sample, such as a sample from a host, or an environmental or industrial sample such as a water sample or a soil sample, and sequencing the organism nucleic acid. The method comprises quantifying the amount of organism based on the amount of the sequenced organism nucleic acid sequence. In particular embodiments the method comprises detecting and quantifying the presence of a non-host organism in a host. Quantifying the amount of the organism may comprise comparing the amount of the sequenced organism nucleic acid sequence to an amount of the one or more nucleic acid sequences sequenced from the one or more reference organisms.

In a further embodiment the method does not require amplification of the organism (such as the non-host organism) nucleic acids. In another embodiment, the organism nucleic acid is not extracted or purified from the sample prior to sequencing.

In another embodiment where the method comprises use of a sample obtained from a host, the method comprises substantial depletion of the host cells from the sample obtained from the host. In a further embodiment, the method comprises addition of a known quantity of at least one reference organism to the sample. The reference organism may be an organism not typically found in the type of sample assayed, such as an environmental or host sample. The reference organism may be a rare organism not typically found in the type of sample assayed, such as an environmental or host sample. Where the sample is a host sample, the reference organism is a non-host organism.

In an embodiment, the method comprises identifying the sequenced nucleic acid sequence as specific to a particular organism. The method may comprise identifying a sequenced non-host nucleic acid sequence as specific to a particular non-host organism. The identification may be achieved by comparison with a database of example organism nucleic acid sequences such as a database of example non-host organism nucleic acid sequences. In a further embodiment, identifying the sequenced nucleic acid sequence as specific to a particular organism comprises sequence alignment with one or more example organism nucleic acid sequences. This alignment may be conducted over the organism nucleic acid entire length.

In some embodiments the one or more example organism nucleic acid sequences comprises a plurality of example organism nucleic acid sequences such as a plurality of example non-host organism nucleic acid sequences. In this case, identifying the sequenced nucleic acid sequence as specific to the organism may further comprise determining a most likely example organism nucleic acid sequence based on relative mapping metrics and/or homology of the nucleic acid sequence with the example organism nucleic acid sequences. This allows the comparison of the probability of various different organisms being present in the sample.

In some embodiments, determining the most likely example organism nucleic acid sequence comprises, in a case where the homology of the sequenced organism nucleic acid sequence with the example organism nucleic acid sequences is similar for a plurality of the example organism nucleic acid sequences, determining the most likely example organism nucleic acid sequence based on ratios of the plurality of the example organism nucleic acid sequences determined as the most likely example organism nucleic acid sequence from sequence alignment of others of the sequenced nucleic acid sequences. This can help to distinguish the case where the sample contains a plurality of highly homologous organisms from the case where an error has occurred in sequencing the organism nucleic acids.

In some embodiments, the method further comprises identifying position-specific sequence differences between the sequenced nucleic acid sequences and the corresponding most likely example organism nucleic acid sequence, optionally wherein the position-specific sequence differences comprise at least one of sequence polymorphisms, insertions, and deletions. The identified position-specific sequence differences may be weighted using error data representing a likelihood of sequencing errors in the sequencing of the at least one sequenced organism nucleic acid sequence. This further enables distinguishing sequencing errors from the presence of highly homologous organisms.

In some embodiments, the method further comprises calculating a frequency measure for one or more of the position-specific sequence differences representing the frequency of the position-specific sequence differences across plural of the sequenced organism nucleic acid sequences in the sequencing data. In some embodiments, the method further comprises calculating a probability of the presence of a plurality of highly homologous organisms based on the frequency measures, optionally further comprising calculating a relative ratio between the highly homologous organisms. If the same position-specific difference occurs with high frequency, this may indicate the presence of plural highly-homologous organisms. Calculating the relative ratio may also allow comparison with known ratios of such highly-homologous organisms to further verify the presence of multiple different types of organism.

In some embodiments, the method further comprises identifying one or more plasmids and/or phages in the sample based on the sequencing data, optionally by comparison of the sequenced organism nucleic acid sequences (such as non-host organism nucleic acid sequences) with one or more example plasmid and/or phage nucleic acid sequences.

In one embodiment the non-host organism is a pathogen. In a further embodiment, the detection and quantification of the non-host organism identifies a pathogen most likely responsible for an infection or disease in the host. The disease may be a systemic infection, a local infection, a urinary tract infection, an infection of the blood, digestive tract infection, a central nervous system infection, a cardiovascular infection, an intro-abdominal infection, a respiratory infection and/or a skin infection. The method may further comprise selecting an agent suitable to treat the pathogen such as an antibiotic or an antifungal.

In some embodiments, the method further comprises identifying one or more antimicrobial resistance genes in the sequenced non-host organism nucleic acid sequences, optionally by sequence alignment of the sequenced non-host organism nucleic acid sequences to one or more example antimicrobial resistance gene sequences. This can help in selecting an appropriate agent for treatment of infection where the non-host organism is a pathogen.

In a further embodiment, provided are methods for monitoring the effectiveness of a treatment of a disease or infection associated with a pathogen in a host, wherein the method comprises detecting and quantifying the pathogen and determining whether the treatment decreases the quantity of the pathogen in a sample from the host.

In some embodiments, the method further comprises estimating a probability of relapse and/or reinfection of the host by the non-host organism based on the amount of the non-host organism.

The invention further provides methods for treating a disease or an infection associated with a pathogen in a host. The method comprises detecting and quantifying the pathogen and administering an agent suitable to treat the pathogen. The agent may be an antibiotic or an antifungal.

The invention also provides a kit which comprises a means for depleting cells from a sample (such as host cells from a sample from a host), one or more reference organisms (such as non-host organisms) in known quantities, and a means for generating a sequence library from nucleic acids (such as non-host nucleic acids).

The invention also provides a method for detecting and quantifying the presence of an organism in a sample, wherein the method comprises: determining an amount of sequenced organism nucleic acid sequence in the sample using sequencing data comprising at least one sequenced organism nucleic acid sequence obtained from sequencing organism nucleic acid from the sample; and quantifying an amount of the organism based on the amount of the sequenced organism nucleic acid sequence. The invention also provides a method for detecting and quantifying the presence of a non-host organism in a host based on a sample obtained from the host, wherein the method comprises: determining an amount of sequenced non-host organism nucleic acid sequence in the sample using sequencing data comprising at least one sequenced non-host organism nucleic acid sequence obtained from sequencing non-host organism nucleic acid from the sample; and quantifying an amount of the non-host organism based on the amount of the sequenced non-host organism nucleic acid sequence.

The method may be a computer-implemented method. The method may be run on generic computing means and may use sequencing data obtained at an earlier time or at another location. The invention also provides a computer program comprising instructions which, when the program is executed by a computer, cause the computer to carry out the method. The invention also provides a computer-readable storage medium comprising instructions which, when executed by a computer, cause the computer to carry out the method.

In some embodiments, quantifying the amount of the organism further comprises determining a recovery ratio. The recovery ratio is a ratio of the amount of the nucleic acid sequence sequenced from the one or more reference organisms to an expected amount of nucleic acid in the sample from the one or more reference organisms. The expected amount is based on an amount of the reference organism added to the sample prior to sequencing and, optionally, on a genome length of the one or more reference organisms. The recovery ratio may be a ratio of the amount of the nucleic acid sequence sequenced from the one or more reference non-host organisms to an expected amount of nucleic acid in the sample from the one or more reference non-host organisms. The expected amount is based on an amount of the reference non-host organism added to the sample prior to sequencing and, optionally, on a genome length of the one or more reference non-host organisms. Using a recovery ratio allows the method to compensate for imperfections in the sequencing data that may lead to not all of the nucleic acid from the organism being correctly sequenced and identified.

In some embodiments, quantifying the amount of the organism, such as a non-host organism, comprises estimating a total amount of the organism nucleic acid using the amount of the organism nucleic acid sequence and the recovery ratio, and estimating the amount of the organism in the sample based on the total amount of the organism nucleic acid and a genome length of the organism. This allows the method to account for differences in the length of the genome between organisms, such as non-host organisms.

In some embodiments, quantifying the amount of the organism may comprise calculating the percentage of the total organisms in the sample that the organism makes up in the sample. In some embodiments, quantifying the amount of the organism may comprise calculating a cell number of the organism in the sample. Quantifying the amount of the organism may additionally or alternatively comprise calculating the percentage of the total number of organisms in the sample made up by the organism. Calculating the percentage of the organism may involve calculating the percentage of nucleic acid sequence reads associated with a particular organism out of the total nucleic acid reads for all organisms in the sample. The total nucleic acid reads may include reads only associated with identified organisms or reads associated with both identified organisms and unknown organisms. In some embodiments, calculating the percentage of the organism may involve calculating the percentage of nucleic acid sequence reads associated with a particular non-host organism out of the total nucleic acid reads for all non-host organisms in the sample. Calculating the cell number may involve determining a recovery ratio of the nucleic acids sequenced by the method. The recovery ratio may be a ratio of the amount of the nucleic acid sequence sequenced from the one or more reference organisms (such as reference non-host organisms) to an expected amount of nucleic acid in the sample from the one or more reference organisms (such as reference non-host organisms). The expected amount is based on an amount of the reference organism (such as reference non-host organism) added to the sample prior to sequencing and, optionally, on a genome length of the one or more reference organisms (such as reference non-host organisms). The expected amount of nucleic acid may be correlated back to the known cell number of reference organism added during the method for example at S110 in FIGS. 1 and 2.

The invention also provides an apparatus for detecting and quantifying the presence of an organism in a sample, the apparatus comprising: a determining unit configured to determine an amount of sequenced organism nucleic acid sequence in the sample using sequencing data comprising at least one sequenced organism nucleic acid sequence obtained from sequencing organism nucleic acid from the sample; and a quantifying unit configured to quantify an amount of the organism based on the amount of the sequenced organism nucleic acid sequence. The invention further provides an apparatus for detecting and quantifying the presence of a non-host organism in a host based on a sample obtained from the host, the apparatus comprising: a determining unit configured to determine an amount of sequenced non-host organism nucleic acid sequence in the sample using sequencing data comprising at least one sequenced non-host organism nucleic acid sequence obtained from sequencing non-host organism nucleic acid from the sample; and a quantifying unit configured to quantify an amount of the non-host organism based on the amount of the sequenced non-host organism nucleic acid sequence.

### BRIEF DESCRIPTION OF FIGURES

The invention will be further described by way of non-limitative example with reference to the accompanying drawings in which:

FIG. 1 is a flowchart illustrating a method for obtaining sequencing data of an organism from a sample

FIG. 2 is a flowchart illustrating a method for obtaining sequencing data of a non-host organism from a sample obtained from the host;

FIG. 3 is a flowchart illustrating steps related to estimating the amount of the non-host organism.

FIG. 4 is a flowchart illustrating steps related to identifying the non-host organism.

FIG. 5 is a graph showing the absolute cell number of the mixed population of bacteria and fungi in Zymo D6300 measured by the method of Example 1. From left to right the mixed bacteria and fungi species are Pseudomonas aeruginosa, Escherichia coli, Salmonella enterica, Lactobacillus fermentum, Enterococcus faecalis, Staphylococcus aureus, Listeria monocytogenes, Bacillus subtilis, Saccharomyces cerevisiae and Cryptococcus neoformans. For each species there are five bars plotted, the left most bar (black bar) indicates the known absolute cell count if the species from the Zymo D6300 solution. The remaining four bars (four grey bars) indicate the measured absolute cell count for each species across four independent repeats 1-4 with each of repeat represented as a single bar.

FIG. 6 is a graph plotting expected titres of Zymo D6300 against measured cell titres by the method of Example 1. Each datapoint for Pseudomonas aeruginosa, Escherichia coli, Salmonella enterica, Lactobacillus fermentum, Enterococcus faecalis, Staphylococcus aureus, Listeria monocytogenes, Bacillus subtilis, Saccharomyces cerevisiae and Cryptococcus neoformans falls within the margin of 2× under or over reporting as illustrated by the top and bottom lines. The middle dashed line represents 100% concordance with the estimated and measured value.

FIG. 7 is a graph showing the average cell number of the mixed population of bacteria and fungi in serial dilutions of Zymo D6300 measured by the method of Example 1. The absolute cell count for each serial dilution was measured and then multiplied by the dilution factor to calculate the cell number of the original 1 in 2.5 dilution of Zymo D6300. Each serial dilution was measured with four independent repeats and then the calculated. From left to right the mixed bacteria and fungi species are Pseudomonas aeruginosa, Escherichia coli, Salmonella enterica, Lactobacillus fermentum, Enterococcus faecalis, Staphylococcus aureus, Listeria monocytogenes, Bacillus subtilis, Saccharomyces cerevisiae and Cryptococcus neoformans. For each species there are six bars plotted, the left most bar (black bar) indicates the known absolute cell counts from the Zymo D6300 solution at a 1 in 2.5 dilutions. The remaining bars indicate the calculated absolute cell count for each species measured from the serial dilution multiplied by the dilution factor for dilutions 1 in 2.5, 1 in 25, 1 in 250, 1 in 2500 and 1 in 25000 which correspond to bars 2 to 6 going from left to right for each species.

FIG. 8 is a plot of expected titres of serial dilutions of E. coli against measured cell titres by the method of Example 1. Each data point of the E. coli dilution falls within the margin of 10× under or over reporting as illustrated by the top and bottom lines. The middle dashed line represents 100% concordance with the estimated and measured value.

FIG. 9 shows ten independent replicates of fresh, sterile ultrapure water analysed by the method illustrated in example 1. The quantitative analysis showed that 9/10 samples returned a low diversity collection of species (>5 mapped reads/species) primarily consisting of Sphingomonas koreensis, Cutibacterium acnes, Pseudomonas stutzeri and Pseudomonas aeruginosa. In FIG. 9 the y-axis of the table lists the following species and subheadings* in descending order: Cutibacterium acnes, Sphingomonas koreensis, Pseudomonas stutzeri, Pseudomonas aeruginosa, Methylobacterium phyllosphaerae, Total reads*, Total cells*, Number of spike reads*, Allobacillus halotolerans and Imtechella halotolerans. In FIG. 9 the x-axis of the table lists Replicates 1-10.

FIG. 10 is a comparison of urinary profiles from 23 healthy female donors. Microbiomes appear to cluster into four major categories dominated by different bacterial species, Gardnerella vaginalis with Fannyhessia vaginae, Lactobacillus crispatus, Lactobacillus iners or Lactobacillus jensenii. In FIG. 10 the y-axis of the table lists the following species in descending order: Gardnerella vaginalis, Fannyhessea vaginae, Bifidobacterium breve, Corynebacterium aurimucosum, Oligella urethralis, Alloscardovia omnicolens, Corynebacterium riegelii, Streptococcus periodonticum, Ezakiella massiliensis, Corynebacterium ureicelerivorans, Anaerococcus mediterraneensis, Corynebacterium tuberculostearicum, Fastidiosipila sanguinis, Peptoniphilus harei, Campylobacter ureolyticus, Corynebacterium imitans, Corynebacterium amycolatum, Trueperella pyogenes, Finegoldia magna, Lawsonella clevelandensis, Corynebacterium jeikeium, Streptococcus anginosus, Actinotignum schaalii, Anaerococcus obesiensis, Anaerococcus vaginalis, Corynebacterium glaucum, Porphyromonas asaccharolytica, Corynebacterium sanguinis, Staphylococcus pettenkoferi, Bifidobacterium dentium, Tessaracoccus timonensis, Corynebacterium kefirresidentii, Staphylococcus epidermidis, Veillonella atypica, Peptoniphilus ivorii, Aerococcus urinae, Fusobacterium canifelinum, Fusobacterium nucleatum, Corynebacterium humireducens, Lactobacillus iners, Aerococcus christensenii, Mobiluncus curtisii, Prevotella intermedia, Fusobacterium nucleatum, Pseudomonas oryzihabitans, Prevotella melaninogenica, Fusobacterium gonidiaformans, Berryella intestinalis, Corynebacterium simulans, Parvimonas micra, Corynebacterium striatum, Ureaplasma urealyticum, Olsenella uli, Lactobacillus crispatus, Lactobacillus jensenii, Limosilactobacillus vaginalis, Lactobacillus gasseri, Streptococcus pasteurianus, Limosilactobacillus fermentum, Streptococcus urinalis, Enterococcus faecalis, Veillonella parvula, Streptococcus agalactiae, Staphylococcus hominis, Staphylococcus haemolyticus, Lactobacillus rhamnosus, Moraxella osloensis, Anaerococcus prevotii, Corynebacterium glucuronolyticum and Rothia amarae. In FIG. 10 the x-axis of the table lists from left to right Donor 51, Donor 45, Donor 29, Donor 43, Donor 46, Donor 24, Donor 34, Donor 23, Donor 33, Donor 82, Donor 37, Donor 31, Donor 41, Donor 28, Donor 48, Donor 26, Donor 39, Donor 40, Donor 27, Donor 30, Donor 22, Donor 50 and Donor 42. Scale of cells/mL lists in descending order 10,000,000; 1,000,000; 100,000; 10,000; 1,000; 100 and 10

FIG. 11A-C shows urine (top) and vaginal swabs (bottom) microbiome profiles from three healthy control female donors taken over a course of five weeks. The five separate sequential weekly assays spanning a full menstrual cycle are shown for each. Estimated cells/mL of urine or cells/swab are shown for each species at each time-point. In FIG. 11A the y-axis of the table lists the following species and subheadings* in descending order: Top panel*, Lactobacillus iners, Limosilactobacillus vaginalis, Gardnerella vaginalis, Corynebacterium simulans, Corynebacterium kefirresidentii, Finegoldia magna, Streptococcus ruminantium, Bottom panel*, Lactobacillus iners, Limosilactobacillus vaginalis, Gardnerella vaginalis, Corynebacterium simulans, Corynebacterium kefirresidentii, Pseudomonas aeruginosa and Methylobacterium durans. In FIG. 11A the x-axis of the table lists from left to right Week 1, Week 2, Week 3, Week 4, (break for period) and Week 5. In FIG. 11B the y-axis of the table lists the following species and subheadings* in descending order: Top panel*, Lactobacillus crispatus, Gardnerella vaginalis, Lactobacillus iners, Limosilactobacillus vaginalis, Fannyhessea vaginae, Peptoniphilus harei, Finegoldia magna, Prevotella intermedia, Aerococcus christensenii, Lawsonella clevelandensis, Bottom panel*, Lactobacillus crispatus, Gardnerella vaginalis, Lactobacillus iners, Limosilactobacillus vaginalis, Fannyhessea vaginae, Aerococcus christensenii, Lactobacillus jensenii and Streptococcus urinalis. In FIG. 11C the x-axis of the table lists from left to right Week 1, Week 2, (break for period), Week 3, Week 4 and Week 5. In FIG. 11C the y-axis of the table lists the following species and subheadings* in descending order: Top panel*, Lactobacillus crispatus, Gardnerella vaginalis, Lactobacillus iners, Limosilactobacillus vaginalis, Fannyhessea vaginae, Lactobacillus jensenii, Lactobacillus gasseri, Aerococcus christensenii, Streptococcus urinalis, Bottom panel*, Lactobacillus crispatus, Gardnerella vaginalis, Lactobacillus iners, Limosilactobacillus vaginalis, Fannyhessea vaginae, Lactobacillus jensenii, Lactobacillus gasseri, Aerococcus christensenii and Streptococcus urinalis. In FIG. 11c the x-axis of the table lists from left to right Week 1, Week 2, Week 3, (break for period), Week 4 and Week 5. For FIGS. 11A-C the scale of cells/swab (Vaginal Swab) or cells/mL (Urine) lists in descending order 10,000,000; 1,000,000; 100,000; 10,000; 1,000; 100 and 10.

FIG. 12 shows a comparison of vaginal swab profiles from healthy female donors. The microbiomes appear to cluster into four major categories dominated by different bacterial species, Gardnerella vaginalis with Fannyhessia vaginae, Lactobacillus crispatus, Lactobacillus iners or Lactobacillus jensenii. In FIG. 12 the y-axis of the table lists the following species in descending order: Gardnerella vaginalis, Fannyhessea vaginae, Corynebacterium aurimucosum, Bifidobacterium breve, Corynebacterium tuberculostearicum, Corynebacterium riegelii, Corynebacterium amycolatum, Corynebacterium ureicelerivorans, Corynebacterium imitans, Streptococcus periodonticum, Alloscardovia omnicolens, Finegoldia magna, Corynebacterium jeikeium, Fastidiosipila sanguinis, Lawsonella clevelandensis, Peptoniphilus harei, Anaerococcus mediterraneensis, Staphylococcus pettenkoferi, Streptococcus anginosus, Anaerococcus obesiensis, Actinotignum schaalii, Anaerococcus vaginalis, Staphylococcus epidermidis, Anaerococcus prevotii, Peptoniphilus ivorii, Bifidobacterium dentium, Cutibacterium granulosum, Cutibacterium avidum, Cutibacterium acnes, Staphylococcus capitis, Corynebacterium nuruki, Berryella intestinalis, Corynebacterium xerosis, Aerococcus urinae, Staphylococcus haemolyticus, Lactobacillus iners, Aerococcus christensenii, Corynebacterium urealyticum, Corynebacterium glucuronolyticum, Streptococcus intermedius, Pseudomonas oryzihabitans, Corynebacterium glaucum, Schaalia cardiffensis, Corynebacterium minutissimum, Pseudomonas psychrotolerans, Prevotella melaninogenica, Sneathia vaginalis, Mycoplasma hominis, Corynebacterium striatum, Prevotella jejuni, Lactobacillus rhamnosus, Corynebacterium kefirresidentii, Corynebacterium simulans, Corynebacterium kroppenstedtii, Mobiluncus curtisii, Corynebacterium singular, Parvimonas micra, Methylobacterium oryzae, Lactobacillus crispatus, Lactobacillus jensenii, Streptococcus urinalis, Limosilactobacillus vaginalis, Lactobacillus gasseri, Staphylococcus hominis, Limosilactobacillus fermentum, Streptococcus pasteurianus, Streptococcus constellatus, Corynebacterium vitaeruminis, Methylobacterium phyllosphaerae, Pseudomonas aeruginosa, Moraxella osloensis, Veillonella parvula, Lactobacillus ultunensis and Methylobacterium durans. In FIG. 12 the x-axis of the table lists from left to right Donor 51, Donor 45, Donor 29, Donor 43, Donor 23, Donor 33, Donor 37, Donor 31, Donor 28, Donor 48, Donor 26, Donor 39, Donor 40, Donor 27, Donor 30, Donor 22 and Donor 50. Scale of cells/swab lists in descending order 10,000,000; 1,000,000; 100,000; 10,000; 1,000; 100 and 10.

FIG. 13 shows comparison of urinary and vaginal microbiome profile composition for the same donor. FIG. 13A. shows a histogram of the percentage of species in each donor that are shared between vaginal and urinary microbiomes (dark blue, bottom section of each bar), specific to vaginal samples (dark green, middle section of each bar) or specific to urine samples (light green, top section of each bar). Where species were found at >1,000 cells/swab any detected level in urine for the same species was scored as a match. FIG. 13B is a graph plotting estimated cell count from vaginal swabs against estimated cell count from urine sample from donor 30 to show an examples of 100% concordance between all measured species (>1,000 cells) in vaginal and urinary microbiomes.

FIG. 14 shows a comparison of bacterial urinary profiles from 18 healthy control male donors. Only species detected at >1,000 cells/mL are shown. 9/18 samples failed to record any species above >1,000 cells/mL. In FIG. 14 the y-axis of the table lists the following species in descending order: Aerococcus christensenii, Haemophilus parainfluenzae, Streptococcus mitis, Streptococcus pseudopneumoniae, Streptococcus gwangjuense, Corynebacterium tuberculostearicum, Peptoniphilus harei, Streptococcus pneumoniae, Finegoldia magna, Anaerococcus mediterraneensis, Campylobacter ureolyticus, Corynebacterium glucuronolyticum, Anaerococcus obesiensis, Fusobacterium nucleatum, Actinotignum schaalii, Gemella haemolysans, Alloscardovia omnicolens, Prevotella jejuni, Prevotella intermedia, Gardnerella vaginalis, Mobiluncus curtisii, Serratia ureilytica, Serratia marcescens, Serratia nematodiphila, Aerococcus urinae, Ezakiella massiliensis, Lactobacillus iners, Streptococcus periodonticum, Veillonella atypica, Enterococcus faecalis, Staphylococcus haemolyticus and Cutibacterium acnes. In FIG. 14 the x-axis of the table lists from left to right Donor 1 to 18. Scale of cells/mL lists in descending order 10,000,000; 1,000,000; 100,000; 10,000; 1,000; 100 and 10.

FIG. 15 shows a cladogram displaying SNP based relationship between consensus builds of Lactobacillus crispatus from urine and vaginal swab samples of the same donors with comparison to 8 ‘Vaginal strains’ and 5 ‘Gut’ strain references taken from Zheng et al. Vaginal and urine consensus references from the same individual are highlighted in the same colour.

FIG. 16 shows a comparison of epithelial vulva and urinary microbiome profiles from ten donors. Samples were collected after using a sterile intimate wipe. Key indicator species, Finegoldia magna and Peptoniphilus harei, are depleted or absent in urine samples despite being highly prevalent in epithelial swabs suggesting that epithelial microbiome contamination does not contribute significantly to urine samples. In FIG. 14 the y-axis of the table lists the following species in descending order: Gardnerella vaginalis, Fannyhessea vaginae, Lactobacillus iners, Finegoldia magna, Lactobacillus crispatus, Lactobacillus jensenii, Streptococcus periodonticum, Peptoniphilus harei, Mobiluncus curtisii, Aerococcus christensenii, Lawsonella clevelandensis, Corynebacterium glucuronolyticum, Anaerococcus obesiensis, Anaerococcus vaginalis, Corynebacterium kefirresidentii, Corynebacterium tuberculostearicum, Actinomyces naeslundii, Streptococcus anginosus, Anaerococcus prevotii, Streptococcus urinalis, Streptococcus constellatus, Cutibacterium acnes, Corynebacterium jeikeium, Staphylococcus hominis, Alloscardovia omnicolens, Corynebacterium atypicum and Dermabacter jinjuensis. In FIG. 16 the x-axis of the table lists from left to right Donor 84, Donor 83, Donor 86, Donor 85, Donor 97, Donor 88, Donor 56, Donor 89, Donor 94 and Donor 5 under the subheadings “Post wipe vulva epithelia swab” and “Post wipe urine analysis”. Scale of cells/mL Urine or total/swab lists in descending order 10,000,000; 1,000,000; 100,000; 10,000; 1,000; 100 and 10.

FIG. 17 shows a comparison of bacterial profiles from urethral swab and urine samples for 7 healthy male controls. Kit-ome associated species are shown in red. In FIG. 17 the y-axis of the table lists the following species in descending order: Cutibacterium acnes, Sphingomonas koreensis, Lactobacillus gasseri, Alloscardovia omnicolens, Corynebacterium tuberculostearicum, Anaerococcus prevotii, Corynebacterium aurimucosum, Limosilactobacillus reuteri, Haemophilus haemolyticus, Haemophilus influenzae, Staphylococcus hominis, Staphylococcus epidermidis, Lactobacillus crispatus, Finegoldia magna, Staphylococcus saprophyticus, Peptoniphilus harei, Corynebacterium glucuronolyticum, Escherichia coli, Corynebacterium kefirresidentii, Streptococcus agalactiae, Streptococcus periodonticum, Fannyhessea vaginae, Gardnerella vaginalis, Lactobacillus iners, Streptococcus gwangjuense, Streptococcus mitis, Streptococcus oralis, Streptococcus pseudopneumoniae, Corynebacterium simulans, Staphylococcus haemolyticus and Corynebacterium ureicelerivorans. In FIG. 17 the x-axis of the table lists from left to right Donor 5.1, Donor 5.2, Donor 24, Donor 25.1, Donor 25.2, Donor 54 and Donor 62 under the subheadings “Post-Wipe Urethral Swab Results” and “Post-Wipe Urine Results”. Scale of cells/mL Urine lists in descending order 10,000,000; 1,000,000; 100,000; 10,000; 1,000; 100 and 10.

### DETAILED DESCRIPTION

It is to be understood that different applications of the disclosed methods may be tailored to the specific needs in the art. It is also to be understood that the terminology used herein is for the purpose of describing particular embodiments of the invention only, and is not intended to be limiting. In addition as used in this specification and the appended claims, the singular forms “a”, “an”, and “the” include plural referents unless the content clearly dictates otherwise. Thus, for example, reference to “an organism” includes two or more such organisms, and the like. All publications, patents and patent applications cited herein, whether supra or infra, are hereby incorporated by reference in their entirety.

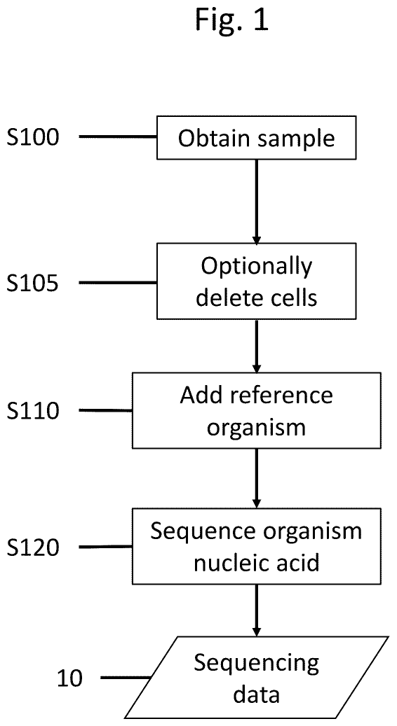

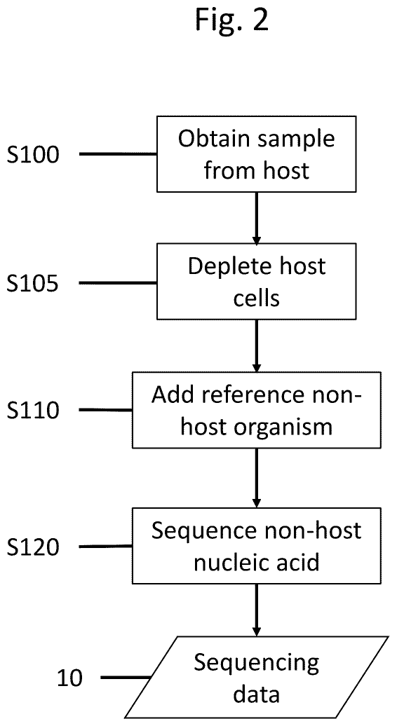

Methods will be described below for detecting and quantifying the presence of an organism in a sample, such as a non-host organism in a host based on a sample obtained from the host. The detecting and quantifying is carried out based on sequencing of organism nucleic acid sequences in the sample to obtain sequencing data 10. As shown in FIGS. 1 and 2, in some embodiments, the method may comprise steps relating to obtaining S100 the sample and sequencing S120 the organism nucleic acid sequences in the sample. In other embodiments, the detecting and quantifying may be carried out based on sequencing data 10 obtained by sequencing the nucleic acid sequences in the sample at an earlier time. In the case where the method uses sequencing data 10 obtained at an earlier time, the method may be entirely implemented using generic computing means adapted to carry out the steps of the method. Detecting and quantifying the presence of an organism in a sample may comprise calculating the absolute cell number of the organism in the sample.

### Organism

Any organism may be detected and quantified, provided that nucleic acids of the organism can be obtained or derived. The organism will typically be non-mammalian. The organism may be for instance a microorganism or a parasite such as a bacteria, fungi, archaea, protozoa, parasite, eukaryotic parasite, virus or bacteriophage. Where the sample is from a host, the organism will typically be a non-host organism. The non-host organism will typically be non-mammalian. The non-host organism may be for instance a microorganism or a parasite such as a bacteria, fungi, archaea, protozoa, parasite, eukaryotic parasite, virus or bacteriophage.

### Sample

Any sample may be used for detection and quantification of an organism, provided that nucleic acids of the organism can be obtained or derived from the sample. The sample may be for instance an environmental or industrial sample, a reference sample or a clinical sample. An environmental sample may be a water sample, a soil sample, an air sample, a biological sample or a waste sample. An industrial sample may be a food, feed or drink sample. A reference sample may be a blend of known organisms, a single culture of a known organisms or a sterile solution.

Where the methods of the invention are used for diagnosis of a disease by detection and quantification of nucleic acids of the organism, the sample is commonly a clinical sample, for example a sample obtained from a patient suspected of having, or having the disease. Suitable types of clinical sample vary according to the particular type of disease or infection that is present, or suspected of being present in a subject. The sample may be a saliva, blood, urine, tissue, mucus, vaginal swab, faeces, semen, spinal fluid, plasma, sputum and/or serum sample. In preferred embodiments, the samples are taken from animal subjects, such as mammalian subjects. The samples will commonly be taken from human subjects, but the present invention is also applicable in general to domestic animals, livestock, birds and fish. For example, the invention may be applied in a veterinary or agricultural setting. In embodiments where the method detects an infection by Alloscardovia omnicolens, Actinotignum schaalii, Escherichia coli, Klebsiella pneumoniae, Enterococcus faecalis, Proteus mirabilis, Pseudomonas aeruginosa, Staphylococcus agalactiae, Staphylococcus saprophyticus, Staphylococcus epidemidis, Gardnerella vaginalis, Finegoldia magna, Corynebacterium riegelii, Oligella urethralis, the sample is preferably a urine sample. In embodiments where the method detects a pathogen associated with urinary tract infections such as Alloscardovia omnicolens, Actinotignum schaalii, Escherichia coli, Klebsiella pneumoniae, Enterococcus faecalis, Proteus mirabilis, Pseudomonas aeruginosa, Staphylococcus agalactiae, Staphylococcus saprophyticus, Staphylococcus epidemidis, Gardnerella vaginalis, Finegoldia magna, Corynebacterium riegelii, Oligella urethralis at an amount greater than 10⁵ CFU/ml, a urinary tract infection may be diagnosed. In embodiments where the method detects a pathogen associated with urinary tract infections such as Alloscardovia omnicolens, Actinotignum schaalii, Escherichia coli, Klebsiella pneumoniae, Enterococcus faecalis, Proteus mirabilis, Pseudomonas aeruginosa, Staphylococcus agalactiae, Staphylococcus saprophyticus, Staphylococcus epidemidis, Gardnerella vaginalis, Finegoldia magna, Corynebacterium riegelii, Oligella urethralis at a cell number greater than 10⁵ cells/ml, the CFU/ml of the pathogen may be determined (by suitable means known in the art, for example by traditional plating techniques) to further confirm the presence of a urinary tract infection with the relevant pathogen. The urine sample may be taken from a subject having a urine infection. The infection may be present in a patient experiencing pain when urinating or excessively urinating. The urine sample may be obtained after cleaning of the urethral entrance to reduce epithelial sample contamination. In embodiments where the urethral entrance is cleaned prior to urine collection, the cleaning may be conducted through the use of a single-use intimate hygienic wipe.

The sample may be known or suspected to comprise one or more organisms. Typically, the identity and quantity of the organisms is not known. The sample typically comprises one or more nucleic acids which may be DNA or RNA of organism. The nucleic acid may be present in the sample in a suitable form allowing for detection and quantitation according to the invention without amplification.

Typically, a host sample is processed in an appropriate manner to remove host cells or host nucleic acid, such as human cells or human nucleic acid in a human sample. Where the sample is an environmental or industrial sample, the depletion of particular cells (e.g. mammalian or human cells) may be optional.

The removal of host cells or host cell nucleic acids from the sample may also be referred to as depleting the host cells or host cell nucleic acids from the sample. For example, the sample is processed to remove at least 95%, at least 96%, at least 97%, at least 98%, at least 99%, at least 99.5%, and/or at least 99.9% of host cells or host nucleic acid originally present in the sample. Suitable chemicals and reaction conditions are known in the art for removing host cells from a sample. For example, a host cell may be removed from a sample through chemical lysis of the host cells and degradation of released host DNA by enzymatic or chemical means. The skilled person is aware of suitable commercial kits for host cell removal such as HostZERO Microbial DNA Kit from Zymo Research. Removal or depletion of host cells may occur at step S105 in the method as illustrated in FIGS. 1 and 2.

After removal of host cells from the sample the remaining non-host cells are lysed. Non-host cells may include bacterial cells, fungi cells, archaea cells, protozoa cells, and/or other parasite cells. Lysis of the remaining non-host cells may be achieved chemically using protease enzymes such as Proteinase K. Non-host cells may also be lysed in a sample using physical methods such as exposure to heat and/or physical disruption with beads. Chemical and physical methods for non-host cell lysis may be combined for lysis of non-host cells from the sample. In some embodiments, following lysis of non-host cells from the sample there is no extraction, isolation and/or purification of nucleic acids from the sample for the subsequent steps of the method.

### Host

The host may be any host in which a non-host organism may be found, typically an animal. Any animal may be considered a suitable host, such as a mammal. The host will commonly be a human and the non-host organism a bacteria or virus, but the present invention is also applicable for example to domestic animals, zoo animals, and livestock. Animals may include any mammals, reptiles, birds, fish and amphibians. Examples of domestic animals may include dogs, cats, rabbits, hamsters, guinea pigs, gerbils, ferrets, chinchillas, mice, rats, snakes, lizards, and newts. Livestock may include horses, cattle, pigs, sheep, goats, deer, alpacas and poultry.

### Reference Organism

Any suitable reference organism may be used to assist quantitation of the organism. A reference organism may also be referred to as a calibrator organism, or a calibrator species. A reference organism may be a rare organism or a rare species. A rare organism, such as a bacteria, may be an organism that does not colonise or infect the environment and/or host as part of their routine behaviour/life cycle or has only been identified in highly restricted geographical/biophysical locations. Typically, the reference organism is not commonly found in the sample of interest. For example, an organism that is present in less than 5%, less than 2%, less than 1% or less than 0.5% of samples taken from the environment, industrial product or host in question may be considered a rare organism. A suitable reference organism may be an organism not found in any samples obtained from the relevant environment and/or host, or which is not found in the environment of the host. Where the host is a human, suitable reference organisms (rare organisms) may include the bacteria species Imtechella halotolerans, Allobacillus halotolerans and/or Truepera radiovictri. The reference organism (such as rare organism) is typically added as a whole organism to the method, for example as a whole bacterium.

The reference organism (such as rare organism), is typically added to the sample at any appropriate stage, for example after host cell depletion from a host sample such as at step S110 as shown in FIGS. 1 and 2. The reference organism (such as rare organism) is thus selected to ensure that any presence of that organism in the sample is strictly due to the addition of the reference organism (such as rare organism) after the sample has been obtained. The rarity of the reference organism enables a known quantity of the reference organism to be added to the sample directly correlated with the amount of sequenced reference organism nucleic acid sequence. This measure can then be used to accurately quantify the amount of an organism (such as non-host organism) in the sample (such as host sample). The details of this quantification will be discussed in more detail below.

Where the sample is a host sample, the reference organism (such as rare organism) is typically added in a known quantity after the host cell depletion step.

Preferably the reference organism (such as rare organism) is added as a whole organism. The addition of the reference organism (such as rare organism) as a whole organism enables the recovered ratio based on the yield of the rare organism nucleic acid sequences to account for the processing losses associated with preparing and isolating the organism (such as non-host organism) DNA in the method.

### Nucleic Acids

The organism (such as non-host organism) nucleic acid may be any nucleic acid. A nucleic acid is typically a polymer comprising deoxyribonucleic acid (DNA) monomers and/or ribonucleic acid (RNA) monomers. The organism (such as non-host organism) DNA may be chromosomal DNA or may be extrachromosomal DNA such as a plasmid. The organism (such as non-host organism) DNA may be coding or non-coding chromosomal DNA. The organism (such as non-host organism) extrachromosomal DNA may be a plasmid that contains at least one antimicrobial resistance (AMR) gene. AMR genes may provide antibiotic resistance against any antibiotics, such as beta-lactams, trimethoprims and sulphonamides. The skilled person is aware of resources to determine if a nucleic acid corresponds to an AMR gene, including online databases such as ResFinder. The organism (such as non-host organism) RNA may be an mRNA transcript. The organism (such as non-host organism) RNA may be part of an rRNA transcript. The organism (such as non-host organism) DNA may be part of a bacteriophage genome. The organism (such as non-host organism) DNA or RNA may be part of a viral genome. The organism (such as non-host organism) nucleic acid may be a fragment of any nucleic acid as described above such as a fragment of a plasmid DNA or chromosomal DNA. The organism (such as non-host organism) nucleic acid is typically greater than 500 bp, 750 bp, or 1000 bp in length.

Sequence data is obtained for the organism nucleic acid, typically sufficient data to identify a unique nucleic acid sequence to the organism. The skilled person is aware of techniques for interrogating the presence of unique nucleic acid sequences that enable determination of the nucleic acid origin. These unique nucleic acid sequences may be highly divergent between species, particularly closely related species. These unique nucleic acid sequences may correspond to highly variable regions of DNA.

The methods of the invention may comprise use of previously obtained sequence data, or include a step of sequencing an organism (such as non-host organism) nucleic acid. The organism nucleic acid may be sequenced by any known technique. Suitable techniques for sequencing DNA include next generation sequencing methods such as Illumina (Solexa) sequencing, pyrosequencing, ion semiconductor sequencing, sequencing by ligation (SOLID), and third-generation/long-read sequencing (such as nanopore sequencing and PacBio single-molecule real-time (SMRT)). Further DNA sequencing techniques include microscopy based methods (such as atomic force microscope and transmission electron microscopy), micro arrays, mass spectrometry, microfluidic Sanger sequencing, RNA polymerase (RNAP) sequencing, and in vitro virus high-throughput sequencing. Suitable techniques for sequencing RNA include quantitative reverse transcriptase polymerase chain reaction (RT-qPCR).

Preferably, the sequencing method provides a read length of greater than 500 bp, 750 bp, or 1000 bp in length. A particularly preferred sequencing method is nanopore sequencing.

The methods of the invention may comprise the generation of a sequence library. Methods for generating a sequence library are well known in the art and any such method may be used. Typically, once the nucleic acid species of interest (either DNA or RNA) has been obtained, the nucleic acids are fragmented, optionally into particular lengths, and used to generate a library. Once fragmented, the nucleic acid molecules may be modified to have specific adapters added to both ends of the nucleic acid sequence. The adapters may be selected to allow the nucleic acid to be bound to the surface of a reaction vessel and remain immobile while sequencing occurs.

### Sequence Alignment

The obtained or sequenced nucleic acid sequence is analysed to determine the origin of the sequence, typically by determining its identity or similarity to a known sequence. In order to determine the identity or similarity of a sequence, the sequence is typically aligned against a known sequence and the identity or similarity of the two sequences compared and quantified. This comparison and quantification may be described as the percentage similarity or identity, the number of mis-matches or other known scores.

Sequences may be aligned for optimal comparison purposes (e.g., gaps can be introduced in a first sequence for optimal alignment with a second sequence). For the purposes of the invention, the sequences are preferably aligned and the nucleotides at each position are then compared. When a position in the first sequence is occupied by the same nucleotide at the corresponding position in the second sequence, then the nucleotides are identical at that position. The percent identity between the two sequences is a function of the number of identical positions shared by the sequences (i.e., % identity=number of identical positions/total number of positions in the reference sequence×100).

Typically the sequence comparison is carried out over the length of the reference sequence. For example, if the user wished to determine whether a given (“test”) sequence is at least 95% identical to a known sequence, the known sequence would be the reference sequence. To assess whether a sequence is at least 95% identical to a reference sequence, the skilled person would carry out an alignment over the length of the reference sequence, and identify how many positions in the test sequence were identical to those of the reference sequence. If at least 95% of the positions are identical, the test sequence is at least 95% identical to the reference sequence. If the test sequence is shorter than the reference sequence, the gaps or missing positions should be considered to be non-identical positions.

Similarly, to determine whether a “test” sequence is or comprises a sequence that is at least 95% identical to a fragment of the reference sequence, the skilled person would align the test sequence with the reference sequence and identify a contiguous portion of the reference sequence of the required length which best aligns with the test sequence (“reference fragment”). The corresponding portion of the “test” sequence which aligns to the “reference fragment” is the “test fragment”. The skilled person would then calculate the percentage identity between the “test fragment” and the “reference fragment”, using the calculation % identity=(number of positions that are identical between the “test fragment” and the “reference fragment”/the length of the “reference fragment”)×100. For example, to determine whether a “test” sequence is or comprises a sequence that is at least 95% identical to a fragment of at least X nucleic acids of the reference sequence, the skilled person would align the test sequence with the reference sequence, and identify a contiguous X nucleotides portion of the reference sequence which best aligns with the test sequence (in this example, this would be the “reference fragment”). The corresponding portion of the “test” sequence which aligns to the X nucleotides portion of the reference sequence is the “test fragment” in this example. The user then calculates the percentage identity between the “test fragment” and the X nucleotides portion of the reference sequence that aligns to the test fragment (“reference fragment”) as described above, i.e. using the calculation % identity=number of positions that are identical between the “test fragment” and the “reference fragment”/X (the length of the “reference fragment”)×100.

The skilled person is aware of different computer programs that are available to determine the homology or identity between two sequences. For instance, a comparison of sequences and determination of percent identity between two sequences can be accomplished using a mathematical algorithm. In an embodiment, alignments may be performed using global alignment (for example Needleman-Wunsch algorithm) local alignment (for example Smith-Waterman algorithm), pairwise alignment and/or multiple sequence alignment. Preferably, the alignment is performed by the Basic Local Alignment Search Tool (BLAST).

When conducting the sequence analysis using the above mentioned alignment approaches, the skilled person is able to adapt the analysis and algorithm parameters to account for the qualities of the “test” sequence. These qualities include the length of the “test” sequence and the length of any example sequences.

The obtained or sequenced sequence may be aligned against one or more reference sequences from a database. The skilled person is aware of existing databases for aligning the “test” sequence against such as GenBank, the National Institutes of Health sequence database. Alternatively, a custom sequence database may be used. Construction of a custom sequence database may allow for the inclusion of all potential sequences of interest, a feature not always possible with large public sequence databases. There is also the advantage of excluding sequences that are not of interest or reference degeneracy to reduce the generation of irrelevant alignments. In some embodiments, a custom sequence database includes nucleic acid sequences which correspond to unique nucleic acid sequences in organisms (such as non-host organisms) of interest. In a further embodiment, a custom sequence database includes nucleic acid sequences originating from both chromosomal and extrachromosomal DNA of the organisms (such as non-host organisms) of interest. In some embodiments, the custom sequence database includes nucleic acid sequences of AMR genes present on extrachromosomal DNA of the organisms (such as non-host organisms) of interest. In some embodiments the custom sequence database includes nucleic acid sequences of rare organisms (such as rare non-host organisms) that are not typically found in the environment and/or host. In some embodiments where the sample is from a host, the custom sequence database includes nucleic acid sequences of the host organism.

If a nucleic acid sequence cannot be aligned against a sequence in a custom database, any existing database, such as GenBank, the National Institutes of Health sequence database, may be used to cross-check the unidentified nucleic acid sequence. If a nucleic acid sequence cannot be aligned against a sequence in a database or multiple databases this may indicate an organism that doesn't currently have a reference in the database(s).

In another embodiment custom databases may comprise a nucleic acid sequence obtained from a previous non-host sample, such as a patient sample. In another embodiment custom databases may be continually updated with nucleic acid sequence obtained from a previous non-host sample in order to replace nucleic acid sequences of poor quality, expand the number of nucleic acid sequences in the database and/or account for any genetic changes in an organism (such as a non-host organism) including genetic changes through evolution, genetic shift and genetic drift.

### Determining and Quantifying Presence of the Organism

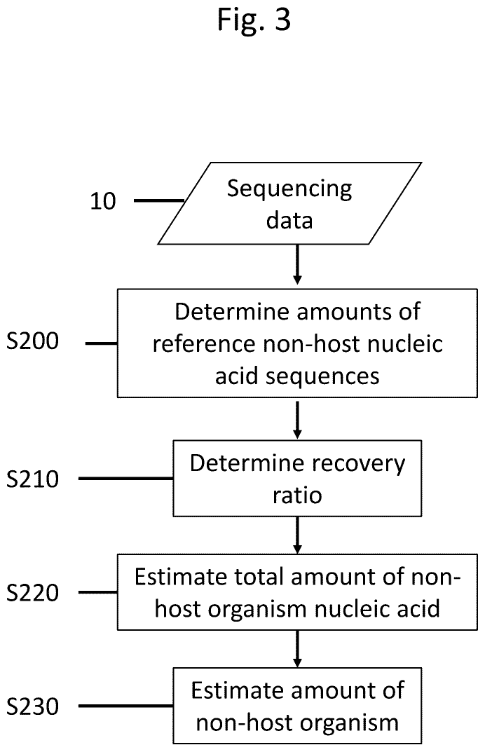

The invention provides a method for detecting and quantifying the presence of an organism in a sample, such as in a host based on a sample obtained from the host. As shown in FIG. 3, the method comprises determining S200 an amount of sequenced organism (such as non-host organism) nucleic acid sequence in the sample using sequencing data 10. The sequencing data 10 comprises at least one sequenced organism (such as non-host organism) nucleic acid sequence obtained from sequencing organism nucleic acid (such as non-host organism) from the sample. The sequencing data 10 may be obtained by obtaining organism (such as non-host organism) nucleic acid from the sample in any suitable manner as described elsewhere, and sequencing at least one organism (such as non-host organism) nucleic acid sequence using the organism nucleic acid. The sequencing may be Nanopore sequencing.

The method comprises quantifying an amount of the organism (such as non-host organism) based on the amount of the sequenced organism (such as non-host organism) nucleic acid sequence. The sequencing data 10 may further comprise one or more nucleic acid sequences sequenced from one or more reference organisms (such as reference non-host organisms) in the sample. The reference organisms may be as described above. As also mentioned above, a known quantity of the reference organism (such as reference non-host organism) may be added S110 to the sample (such as the sample obtained from the host) before it is sequenced to obtain the sequencing data 10. Quantifying the amount of the organism (such as non-host organism) may then comprise comparing the amount of the sequenced organism (such as non-host organism) nucleic acid sequence to the amount of the one or more nucleic acid sequences sequenced from the one or more reference organisms (such as reference non-host organisms). The method may comprise determining that the organism (such as non-host organism) is present in the sample, for example if the amount of the organism (such as non-host organism) is above a predetermined threshold.

The method may comprise repeating the steps of determining an amount of sequenced organism nucleic acid sequence and quantifying an amount of the organism for multiple organisms.

Quantifying the amount of the organism (such as non-host organism) may comprise determining S210 a recovery ratio. The recovery ratio is representative of a proportion of nucleic acid from organisms in the sample that are recovered by the sequencing process. For example, not all nucleic acid from the organisms may be successfully recovered or sequenced, and/or not all of the nucleic acid from the organism that is sequenced may be successfully identified as coming from the correct organism. The latter situation may be particularly the case when “next generation” sequencing techniques are used that fragment the nucleic acid and sequence many small fragments. By adding the known quantity of nucleic acid from the reference organism (such as a reference non-host organism) to the sample, preferably by adding a known number of reference organism (such as a reference non-host organism) cells, and then seeing how much is recovered when sequencing the sample, it can be estimated what proportion of nucleic acid from other organisms in the sample is captured by the sequencing process.

The recovery ratio is a ratio of the amount of the nucleic acid sequence sequenced from the one or more reference organisms (such as reference non-host organisms) to an expected amount of nucleic acid in the sample from the one or more reference organisms (such as reference non-host organism). The recovery ratio Rr may be given by

$$R = \frac{{E}_{r}}{{M}_{r}}$$

where Eᵣ is the expected amount of nucleic acid in the sample from the one or more reference organisms (such as reference non-host organisms), and Mᵣ is the (measured) amount of the nucleic acid sequence sequenced from the one or more reference organisms (such as reference non-host organisms).

The expected amount may be based on an amount of the reference organism (such as reference non-host organism) added to the sample prior to sequencing. For example, the expected amount could be determined based also on a genome length of the one or more reference organisms (such as reference non-host organisms). The expected amount Eᵣ may be given by

$${E}_{r} = {N}_{r} * {L}_{r}$$

where Nᵣ is the amount of the reference organism (such as reference non-host organism) added to the sample prior to sequencing, quantified as the number of cells of the reference organism (such as reference non-host organism) added to the sample, and L, is the genome length of the reference organism (such as reference non-host organism).

Quantifying the amount of the organism (such as non-host organism) may comprise estimating S220 a total amount of the organism (such as non-host organism) nucleic acid using the amount of the organism (such as non-host organism) nucleic acid sequence and the recovery ratio. This uses the recovery ratio to compensate for the proportion of nucleic acid from the organism (such as non-host organism) that may have been lost during processing or incorrectly identified. The total amount of the organism (such as non-host organism) nucleic acid Tₙₕ may be estimated as

$${T}_{n h} = R * {M}_{n h}$$

where Mₙₕ is the amount of the organism (such as non-host organism) nucleic acid sequence. The amount of the organism (such as non-host organism) in the sample can then be estimated S230 based on the total amount of the organism (such as non-host organism) nucleic acid and a genome length of the organism (such as non-host organism). The amount of the organism (such as non-host organism) in the sample, quantified as the number of cells of the organism (such as non-host organism) in the sample Nₙₕ, may be estimated as

$${N}_{nh} = \frac{{T}_{nh}}{{L}_{nh}}$$

where Lₙₕ is the genome length of the organism (such as non-host organism).

Quantifying the amount of the organism (such as non-host organism) may comprise comparing the amount of the sequenced organism (such as non-host organism) nucleic acid sequence of one identified organism to the total amount of sequenced organism (such as non-host organism) nucleic acids for the total sample. The method may comprise calculating the proportion of sequenced nucleic acids associated with a single organism (such as non-host organism) out of the total sequenced nucleic acids. Calculating the proportion of nucleic acid reads may include adjustments for the genome length of the reference organism (such as non-host organism).

### Identifying the Organism

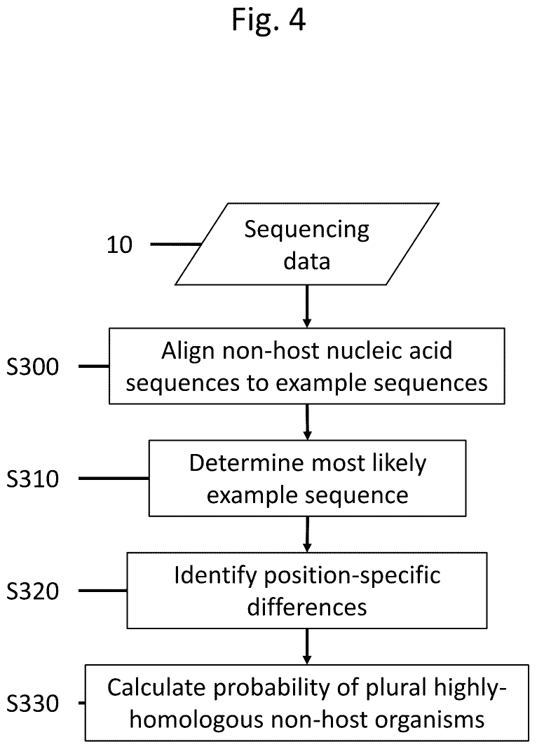

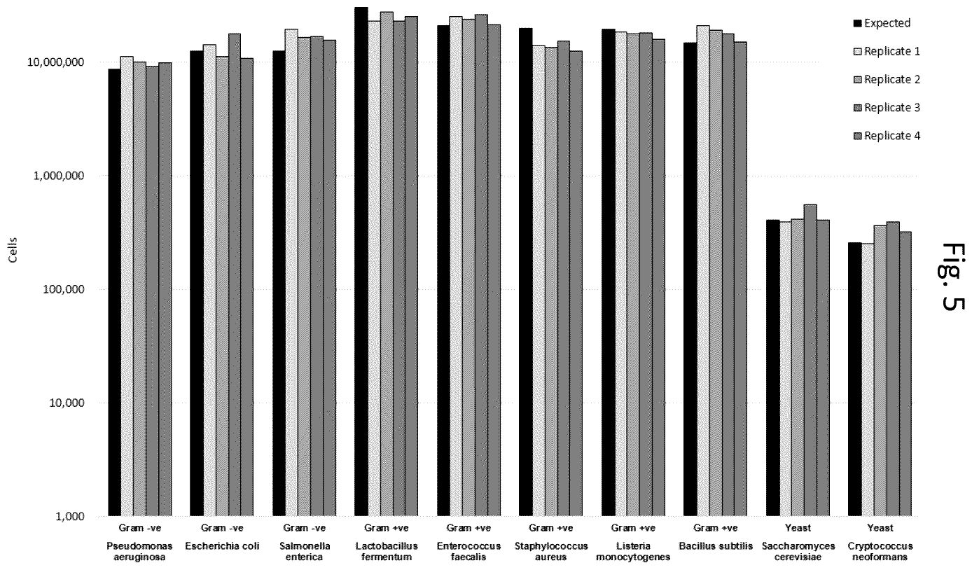

As mentioned above, the method may comprise identifying the sequenced nucleic acid sequence (such as non-host nucleic acid sequence) as specific to a particular organism (such as non-host organism), as illustrated in FIG. 4. The identification may be achieved by comparison with a database of example organism nucleic acid sequences (such as non-host organism nucleic acid sequences), for example using sequence alignment S300 with the one or more example organism nucleic acid sequences (such as non-host organism nucleic acid sequences). Any suitable sequence alignment method may be used, for example the Basic Local Alignment Search Tool (BLAST).

In general, the database of example organism nucleic acid sequences (such as non-host organism nucleic acid sequences) may comprise a plurality of example organism nucleic acid sequences (such as non-host organism nucleic acid sequences). In this case, identifying the sequenced nucleic acid sequence (such as non-host nucleic acid sequence) as specific to the organism (such as non-host organism) may further comprise determining S310 a most likely example organism nucleic acid sequence (such as non-host organism nucleic acid). This determination may be based on relative mapping metrics including level of sequence identity, homology and/or length of match, specific insertions, deletions and/or single nucleotide polymorphisms (SNPs) with respect to the example organism nucleic acid sequences (such as non-host organism nucleic acid sequences). In the event of multiple possible matches to different example reference organism nucleic acids (such as non-host organism nucleic acids), comparison between metrics for the two matches will be used to identify the closest homology for a given nucleic acid sequence (such as non-host nucleic acid sequence). In one embodiment this comparison will use the ‘raw mapping’ scores produced by BLAST to compute a relative measure of the similarity of the homologies for the first and second highest homology matches from the database of example organisms (such as non-host organism) for a given nucleic acid sequence (such as non-host nucleic acid sequence). Where the second mapping raw score, when transformed into a percentage of the first mapping raw score, falls below a user pre-determined threshold it will be assumed that the first match is sufficiently unique to provide a robust identification for the organism (such as non-host organism) of origin. A match may be called when a species is founds at greater than 1000 cells per sample, such as greater than 1000 cells per swab. In an embodiment, where species are found at greater than 1000 cells per sample in one sample may, the match may be used to score a match of any detected level of the same species in a second sample. For example, where species are found at greater than 1000 cells per swab any detected level in urine for the same species may be scored as a match. The first sample and second sample may be from the same host. The first sample and second sample may be different types of samples, for example a saliva, blood, urine, tissue, mucus, vaginal swab, faeces, semen, spinal fluid and/or plasma sample. The method may also comprise calculating, and optionally outputting, a confidence metric representing a level of certainty that the sequenced nucleic acid sequence (such as non-host nucleic acid sequence) originates from the identified organism (such as non-host organism). The confidence metric may be calculated using the relative mapping metrics. The confidence metric may include a minimum number of reads mapping to an organism. The minimum number of reads may be at least 5 mapped reads/species, at least 10 mapped reads/species, at least 15 mapped reads/species, at least 20 mapped reads/species, at least 30 mapped reads/species, at least 40 mapped reads/species or at least 50 mapped reads/species.

In some situations, there may not be a clear most likely corresponding organism (such as non-host organism) in the database. This may be the case, for example, where several genetically similar organisms (such as non-host organisms) are present in the sample. In this case, the homology of the sequenced nucleic acid sequence (such as non-host nucleic acid sequence) with the example organism nucleic acid sequences (such as example non-host organism nucleic acid sequences) may be similar for a plurality of the example organism nucleic acid sequences (such as non-host organism nucleic acid sequences). In such cases, determining S310 the most likely example organism nucleic acid sequence (such as non-host organism nucleic acid sequence) may comprise determining the most likely example organism nucleic acid sequence (such as non-host organism nucleic acid sequence) based on ratios of the plurality of the example organism nucleic acid sequences (such as non-host organism nucleic acid sequences) determined as the most likely example organism nucleic acid sequence (such as non-host organism nucleic acid sequence) from sequence alignment of others of the sequenced nucleic acid sequences (such as non-host nucleic acid sequences).

The others of the sequenced nucleic acid sequences (such as non-host nucleic acid sequences) may be sequenced nucleic acid sequences that have been identified with a high degree of certainty as corresponding to one of the example organism nucleic acid sequences (such as non-host organism nucleic acid sequences). For example, using the previously stated logic, the identification may be considered to have a sufficiently high degree of certainty where the second highest mapping raw score, when transformed into a percentage of the highest mapping raw score, falls below a user pre-determined threshold, thereby rendering the first match sufficiently unique to provide a robust identification for the organism (such as non-host organism) of origin. Ratios of organisms with robust identifications computed in this way would then be used to inform the likely attribution of nucleic acid sequences (such as non-host nucleic acid sequences) sharing the same example organism nucleic acid sequences (such as non-host organism nucleic acid sequences) as the highest homology match and second highest homology match, but where the relative mapping metrics are sufficiently similar to be equal or above the pre-determined threshold. The predetermined threshold may be determined by the user when setting up the method.

The method may also allow for identifying the presence of highly homologous organisms (such as non-host organisms), for example from sub-species, strains or sub-strains of organism. The method may further comprise identifying S320 position-specific sequence differences between the sequenced nucleic acid sequences (such as non-host nucleic acid sequences) and the corresponding most likely example organism (such as non-host organism) nucleic acid sequence. The position-specific sequence differences may comprise at least one of sequence polymorphisms, insertions, and deletions.

The identified position-specific sequence differences may be weighted using error data representing a likelihood of predetermined, technology-specific sequencing errors in the sequencing of the at least one sequenced organism nucleic acid sequence (such as non-host organism nucleic acid sequence). Although individual errors are unpredictable, the types and frequencies of errors are generally well-defined and predictable, and vary between different sequencing technologies. This can allow the method to take account of the likelihood that position-specific differences are due to technology specific sequencing errors, rather than due to the presence of a sub-species, strain or sub-strain of the organism (such as non-host organism).

The method may further comprise calculating a frequency measure for one or more of the position-specific sequence differences. The frequency measure represents the frequency of the position-specific sequence differences across plural of the sequenced organism nucleic acid sequences (such as non-host organism nucleic acid sequences) in the sequencing data. This could, for example, be a proportion of the-organism nucleic acid sequences (such as non-host organism nucleic acid sequences) that contain the same position-specific sequence difference. The frequency measure may be calculated only based on a user-defined threshold level of organism nucleic acid sequences (such as non-host organism nucleic acid sequences) that have been mapped to the part of the genome of the organism (such as non-host organism) that includes the position of the position-specific sequence difference. This may help to avoid giving too low an estimate of the frequency of the position-specific sequence difference by counting nucleic acid sequences that do not have sufficient coverage of the position of the difference as lacking the difference.

The method may further comprise calculating S330 a probability of the presence of a plurality of highly homologous organisms (such as non-host organisms) based on the frequency measures. For example, a higher frequency measure for a particular position-specific sequence difference (or combination of position-specific sequence differences) may correspond to a higher probability of the presence of a plurality of highly homologous organisms (such as non-host organisms). If the frequency measure for the position-specific sequence difference is above a predetermined threshold, such as 10%, optionally 25%, it may be concluded that the prevalence of the position-specific sequence difference is due to a heterogeneous population of an organism (such as non-host organism) having a highly homologous genome to the most similar example organism (such as non-host organism), but constituting separate sub-species, strains or sub-strains. To avoid returning many spurious results, the probability of the presence of a plurality of highly homologous organisms (such as non-host organisms) may also depend on the amount of organism (such as non-host organism) nucleic acid sequences covering the position of the position-specific difference. For example, the method may indicate that the probability of the presence of a plurality of highly homologous organisms (such as non-host organisms) is lower when only very few organism nucleic acid sequences (such as non-host organism nucleic acid sequences) (for example less than 10× coverage, optionally less than 20× coverage) cover the position of the position-specific difference, even if a high proportion of those organism nucleic acid sequences display the position-specific difference. The method may further comprise calculating a relative ratio between the highly homologous organisms (such as non-host organisms). This can allow for the identification of the relative prevalence of the strains or sub-species in the sample from the host.

### Measures for Quantification of an Identified Organism

In a preferred embodiment, the method may calculate a cell number of an organism (such as non-host organism) in the sample by using the number of sequencing reads recovered for the identified organism taking into account the number of sequencing reads recovered for the one or more reference organisms (such as a reference non-host organisms). In an embodiment the sequence reads are not limited to a single gene, such as a 16s RNA gene. The detection and quantification of the organism according to the method may be performed without specific primers for, amplification of and/or sequencing of 16s RNA. In a further embodiment, the sequences reads include reads from the whole genome sequence of the organism. In another embodiment, the method may calculate a cell number of an organism (such as non-host organism) in the sample by using the number of sequencing reads recovered for the identified organism (such as non-host organism) taking into account the recovery ratio calculated from the number of sequencing reads recovered for the one or more reference organisms (such as non-host organism). Calculating the cell number of an organism in a sample enables an informed assessment of the relevance of the amount of the organism present. For example, calculation of the cell number of a non-host organism in a host may aid appropriate medical interventions. For example, a urinary tract infection is usually caused by a single organism that is present in a high concentration, usually greater than 10⁵ CFU/ml (Kass EH 1962 Ann Intern Med Vol. pp. 46-53). Such an assessment may also take into account the origin of the sample obtained from the host. Additionally, in environmental or industrial applications, the cell number of the organism can be compared to threshold acceptable levels for the relevant organism in the environmental or industrial sample in question, e.g. in drinking water or food. In an embodiment, the calculated cell number form the method can be confirmed using traditional plating and culturing techniques from the same original sample as used in the method.

Alternatively, the number of sequencing reads recovered for the identified organism (such as non-host organism) may be used to calculate the percentage of the total number of organisms in the sample made up by the organism. In an embodiment the sequences reads are not limited to a single gene, such as a 16sRNA gene. In a further embodiment, the sequences reads include reads from the whole genome sequence of the organism. Calculating the percentage of the organism may involve identifying multiple organisms in the sample, such as multiple non-host organisms in a sample obtained from a host. Calculating the percentage of the organism may involve identifying sequencing reads that do not correspond with a known organism and determining if these reads are due to imperfections in the sequencing data or sequence reads associated with an unidentified organism. Calculating the percentage of the organism may involve comparing the number of sequencing reads recovered for the identified organism with the total number of sequencing reads recovered. The total number of sequence reads recovered may include the sequencing reads of multiple organisms identified in the sample and sequence reads associated with an unidentified organism. Calculating the percentage of the organism provides relative information on the composition of organisms making up the sample but does not provide absolute numbers for organisms identified. This can complicate the interpretation of results for the impact on the host as the most abundant hit may be e.g. a commensal non-pathogenic organism. The percentage of the organism can also only provide information on the proportional abundance of each identified species and cannot be used to determine if the most abundant hit is present at 100 cells/mL or 1,000,000 cells/mL.

### Genetic Resolution

As mentioned above, the sequence alignment of sequence organism nucleic acid sequences (such as non-host organism nucleic acid sequences) may allow for the identification of the origin of the nucleic acid sequence. For example, the sequences may be identified as corresponding to particular genes, plasmids, or bacteriophages. The method may further comprise identifying one or more plasmids and/or phages (e.g. bacteriophages) in the sample based on the sequencing data. The identifying may be performed by comparison of the sequenced non-host organism nucleic acid sequences with one or more example plasmid and/or phage nucleic acid sequences.

In some embodiments, the method further comprises identifying one or more antimicrobial resistance genes in the sequenced organism nucleic acid sequences (such as non-host organism nucleic acid sequences). This may be particularly helpful in embodiments where the organism is a pathogen and the method comprises selecting an agent suitable to treat the pathogen. If an organism (such as a non-host organism) is determined to be likely to have resistance to particular treatment agents, this can improve the selection of appropriate agents to allow more efficient and effective treatment. The identifying of the antimicrobial resistance genes may be performed by sequence alignment of the sequenced organism nucleic acid sequences to one or more example antimicrobial resistance gene sequences. The example antimicrobial resistance gene sequences may be present in an external database, for example.

### Medical and Diagnostic Applications

The method may be used for any diagnostic and/or therapeutic application. In any diagnostic and/or therapeutic application of the described method typically the organism is a non-host organism, the reference organism is a non-host organism and the sample is obtained from a host. Once the sequence identity of the non-host organism and the amount of non-host organism have been calculated using the methods described above, the skilled person can use the data to assess if the non-host organism has a pathogenic, parasitic, symbiotic or mutualistic relationship with the host organism. This assessment will also take into account the nature of the previously obtained host sample, such as a urinary sample or blood sample. The type of sample of the host organism can be indicative of the type of potential infection and provide information of the colonisation of the non-host organism in the host.

A non-host organism that is identified as a pathogen may be selected from bacteria, fungi, archaea, protozoa, parasite, eukaryotic parasite, virus and bacteriophage. The pathogen identified may be that most likely responsible for an infection or disease in the host. The disease in the host may be a systemic infection, a local infection, a urinary tract infection, an infection of the blood, digestive tract infection, a central nervous system infection, a cardiovascular infection, an intro-abdominal infection, a urogenital tract infection, a genital tract (such as vaginal) infection, a respiratory infection and/or a skin infection. Where the disease is a urinary infection, a method of the invention may comprise detecting and quantitating the cell number of the non-host organism as being at least 10⁵ cells/ml. The method may further comprise detecting and quantitating the cell number of the non-host organism as being at least 10⁵ CFU/ml In some embodiments, the method further comprises selecting an agent suitable to treat the pathogen. The agent may be an antibiotic or an antifungal. Suitable antibiotics and antifungals for treatment of particular pathogens are known in the art.

In some embodiments, where the pathogen identified is a bacteria, the method allows for interrogation of any extrachromosomal DNA present in the bacteria. For example, interrogation of plasmids which may contain AMR genes. Where AMR genes are identified in the bacteria, the method allows the selection of a suitable agent or agents in view of any existing antibiotic resistance, avoiding administration of ineffective agents. For example, the method may be used for the selection of targeted therapies for methicillin-resistant Staphylococcus aureus (MRSA).

In some embodiments, a non-host organism can have either a parasitic or a mutualistic relationship with a host organism depending on the amount of the non-host organism and the location of the non-host organism. For example, several organisms including Alloscardovia omnicolens, Actinotignum schaalii, Escherichia coli, Klebsiella pneumoniae, Enterococcus faecalis, Proteus mirabilis, Pseudomonas aeruginosa, Staphylococcus agalactiae, Staphylococcus saprophyticus, Staphylococcus epidemidis, Gardnerella vaginalis, Finegoldia magna, Corynebacterium riegelii and Oligella urethralis are well documented pathogens for urinary tract infections (UTIs). However, for many of these organisms anatomical context is highly relevant and movement from one anatomical compartment, such as an organ system, to another can change the nature of the host and non-host relationship. When the host and non-host relationship becomes pathogenic this can result in the diagnosis of a UTI. In another example, Staphylococcus aureus can colonise the skin and nasal passage without causing disease in a mutualistic relationship. However, if Staphylococcus aureus enters the blood stream, urinary tract or lungs the relationship can transition into a parasitic relationship with Staphylococcus aureus causing disease within the host. In another example, species of Candida yeast (Candida albicans, Candida glabrata and Candida tropicali) commonly colonise the skin and digestive tract in a mutualistic relationship with the host. When the number of Candida increase beyond a certain threshold Candida can start causing disease and migrate into other regions of the host organism such as the throat and vagina creating an infection. Identifying these non-host organisms and determining if the relationship with the host has transitioned from mutualistic to parasitic requires accurate quantification of the amount of organism in the host in order to determine the likelihood that the non-host organism is now a pathogen causing disease, and is thus assisted by the method of the invention.

As described above, the method comprises detecting and quantifying the presence of a non-host organism in a host based on a sample obtained from the host. Typically, the host will be a human and the sample obtained from a host may be a saliva, blood, urine, tissue, mucus, vaginal swab, faeces, semen, spinal fluid and/or plasma sample. The sample is typically processed to remove host cells or host cell nucleic acid. In some embodiments, the sample will be processed to remove at least 95%, at least 96%, at least 97%, at least 98%, at least 99%, at least 99.5%, and/or at least 99.9% of host cells or host cell nucleic acid originally present in the sample.

After removal of the host cells or host cell nucleic acid the method may comprise addition of a known quantity of at least one reference non-host organism to the sample, such as at S.110 of FIGS. 1 and 2. After removal of the host cells or host cell nucleic, the processed sample may comprise at least 1×10³ cells/mL, at least 1×10⁴ cells per/mL or at least 1×10⁵ cells per/mL non-host organism cells. In an embodiment, the processed sample comprises at least 1×10³ cells/mL non-host organism cells. The nucleic acid mixture is then sequenced using methods known to the skilled person. In an embodiment, the sample is sequenced using nanopore sequencing. Once sequenced, the amount of sequenced non-host organism nucleic acid sequence in the sample is determined using sequencing data comprising at least one sequenced non-host organism nucleic acid sequence obtained from sequencing non-host organism nucleic acid from the sample; and quantifying an amount of the non-host organism based on the amount of the sequenced non-host organism nucleic acid sequence.

Once the identity and amount of at least one non-host nucleic acid have been determined, and optionally other factors including the sample origin and host details are considered, the identified organism can be assessed to determine if the non-host organism is a pathogen most likely responsible for an infection or disease in the host. Once a non-host organism has been identified as most likely responsible for an infection or disease in the host such as a systemic infection, a local infection, a urinary tract infection, an infection of the blood, digestive tract infection, a central nervous system infection, a cardiovascular infection, an intro-abdominal infection, a urogenital tract infection, a genital tract (such as vaginal) infection, a respiratory infection and/or a skin infection, a choice of suitable agent for treatment may be selected. This agents may be an antibiotic or an antifungal. The choice of agent may take into consideration the identification of any AMR genes identified in the sample.

The invention further provides a method of monitoring the effectiveness of a treatment of a disease or infection associated with a pathogen in a host. Each of the above described methods for detecting and quantifying the presence of a non-host organism in a host may be employed in a method of monitoring the effectiveness of a treatment of a disease or infection associated with a pathogen in a host. The method for monitoring the effectiveness of a treatment of a disease or infection associated with a pathogen in a host may comprise determining whether the treatment decreases the quantity of the pathogen in a sample obtained from the host. The method may further comprise detecting and quantifying the presence of a non-host organism at at least two time points during the treatment to calculate the change in quantity of a non-host organism over time and determine whether the treatment decreases the quantity of the pathogen in a sample obtained from the host. The method may further comprise detecting and quantifying the presence of a non-host organism at at least two time points during the treatment to calculate the change in quantity of a non-host organism over time and determine whether the treatment decreases the quantity of the pathogen in a sample obtained from the host at a rate that is deemed an effective treatment of the disease.

The at least two time points may comprise a time point taken before the treatment of a disease or infection associated with a pathogen in a host had commenced, a time point taken at the commencement of treatment for the disease or infection associated with a pathogen in a host, a time point taken within 24 hours of commencement of the treatment for the disease or infection associated with a pathogen in a host, a time point taken within 48 hours of commencement of the treatment of a disease or infection associated with a pathogen in a host, a time point taken within 72 hours of commencement of the treatment of a disease or infection associated with a pathogen in a host, a time point taken before 25% of the treatment course for the disease or infection associated with a pathogen in a host had been completed, a time point taken before 50% of the treatment course for the disease or infection associated with a pathogen in a host had been completed and/or a time point after completion of the treatment course for the disease or infection associated with a pathogen in a host. A second time point may be taken 24 hours after the first time point was taken, 48 hours after the first time point was taken, 72 hours after the first time point was taken, once 25% of the treatment course for the disease or infection associated with a pathogen in a host had been completed and/or once 50% of the treatment course for the disease or infection associated with a pathogen in a host had been completed. Multiple time points may be taken through the treatment of a disease or infection associated with a pathogen in a host. Multiple time points may be taken at regular intervals through the treatment for the disease or infection associated with a pathogen in a host such as hourly intervals, 12 hourly intervals, 24 hourly intervals, 48 hourly intervals, 72 hourly intervals, weekly intervals, fortnightly intervals and/or monthly intervals. In an embodiment, the at least two time points comprise a first time point taken at the commencement of treatment and a second time point after completion of the treatment course for the disease or infection associated with a pathogen in a host. The second time point after completion of the treatment course for the disease or infection associated with a pathogen in a host may be taken 24 hours, 48 hours, 72 hours and/or up to a week after completion of the treatment course.

The method may further comprise estimating a probability of relapse and/or reinfection of the host by the non-host organism based on the amount of the non-host organism. For example, if the amount of the non-host organism remains above a predetermined threshold over several time points, or does not decrease at an expected rate, this may indicate a higher probability of relapse. In some embodiments, the presence of any non-host organism identified as a pathogen after completion of the treatment course for the disease or infection associated with a pathogen in a host indicates relapse and/or reinfection of the host by the non-host organism.

### Kits

The invention further provides a kit comprising components required to carry out the process of the invention. The kit optionally further comprises instructions for use in a method of the invention. The kit may comprise a means for depleting cells from a sample, such as host cells from a sample from a host. The kit may comprise one or more reference organism (such as reference non-host organisms) in known quantities. The kit may comprise a means for generating a sequence library from nucleic acids (such as non-host nucleic acids). In an embodiment, the kit comprises (i) a means for depleting cells from a sample from a sample (such as depleting host cells from a sample from a host); (ii) one or more reference organisms (such as reference non-host organisms) in known quantities; and (iii) a means for generating a sequence library from nucleic acids (such as non-host nucleic acids).

The kit optionally comprises a means for enzymatic digestion, thermal and/or physical disruption means for depleting cells from a sample (such as host cells from a sample from a host). Preferably, the enzymatic digestion is a proteinase K digestion. Preferably, the physical disruption is bead bashing and/or sonication. The kit may further comprise suitable buffers and other factors which are required for enzymatic digestion, bead bashing and/or sonication.

The kit optionally comprises one or more reference organisms (such as reference non-host organisms) in a known quantity wherein the organism is a rare bacterium not typically found in the environment and/or host. In an embodiment, the rare bacterium is selected from Imtechella halotolerans, Allobacillus halotolerans and/or Truepera radiovictri. Preferably the rare bacterium is selected from Imtechella halotolerans and/or Allobacillus halotolerans. A plurality of reference organisms (such as non-host organisms) may be provided in the kit as a mixture, or in separate containers. In an embodiment, the plurality of reference organisms (such as non-host organisms) may be provided in the kit as whole organisms.

The kit optionally comprises a means for generating a sequence library, optionally including one or more of means for fragmenting nucleic acids, adaptors, means for addition of the adaptor molecules and/or a reaction vessel.

The kit may comprise a computer readable program containing databases of and/or organism (such as non-host organisms), including the reference organism (such as reference non-host organism). The kit may comprise a computer readable program for determining an amount of sequenced organism nucleic acid sequence (such as sequences non-host organism nucleic acid sequence) in the sample using sequencing data comprising at least one sequenced organism nucleic acid sequence (such as sequenced non-host organism nucleic acid) sequence obtained from sequencing organism nucleic acid from the sample (such as sequencing non-host organism nucleic acid from the sample obtained from a host); and quantifying an amount of the n organism (such as non-host organism) based on the amount of the sequenced organism nucleic acid sequence (such as sequenced non-host organism nucleic acid sequence).

### Aspects of the Invention

Aspect 1. A method for detecting and quantifying the presence of a non-host organism in a host based on a sample obtained from the host, wherein the method comprises:
determining an amount of sequenced non-host organism nucleic acid sequence in the sample using sequencing data comprising at least one sequenced non-host organism nucleic acid sequence obtained from sequencing non-host organism nucleic acid from the sample; and
quantifying an amount of the non-host organism based on the amount of the sequenced non-host organism nucleic acid sequence.

Aspect 2. The method of aspect 1, wherein
the sequencing data further comprises one or more nucleic acid sequences sequenced from one or more reference non-host organisms in the sample; and
quantifying the amount of the non-host organism based on the amount of the sequenced non-host organism nucleic acid sequence comprises comparing the amount of the sequenced non-host organism nucleic acid sequence to an amount of the one or more nucleic acid sequences sequenced from the one or more reference non-host organisms.

Aspect 3. The method of aspect 2, wherein:
quantifying the amount of the non-host organism further comprises determining a recovery ratio;
the recovery ratio is a ratio of the amount of the nucleic acid sequence sequenced from the one or more reference non-host organisms to an expected amount of nucleic acid in the sample from the one or more reference non-host organisms; and
the expected amount is based on an amount of the reference non-host organism added to the sample prior to sequencing and, optionally, on a genome length of the one or more reference non-host organisms.

Aspect 4. The method of aspect 3, wherein quantifying the amount of the non-host organism comprises:
estimating a total amount of the non-host organism nucleic acid using the amount of the non-host organism nucleic acid sequence and the recovery ratio; and
estimating the amount of the non-host organism in the sample based on the total amount of the non-host organism nucleic acid and a genome length of the non-host organism, optionally wherein the total amount of the non-host organism nucleic acid is used to calculate a cell number of the non-host organism in the sample or calculate a percentage of the culture composition of the non-host organism in the sample.

Aspect 5. The method of any one of aspects 2-4, wherein the one or more reference non-host organisms comprise a rare bacterium not typically found in the host, optionally selected from Imtechella halotolerans and/or Allobacillus halotolerans.

Aspect 6. The method of any one of the preceding aspects, wherein the method comprises identifying the sequenced non-host nucleic acid sequence as specific to a particular non-host organism, optionally by comparison with a database of example non-host organism nucleic acid sequences.

Aspect 7. The method of aspect 6, wherein identifying the sequenced non-host nucleic acid sequence as specific to a particular non-host organism comprises sequence alignment with one or more of the example non-host organism nucleic acid sequences.

Aspect 8. The method of aspect 7, wherein the sequence alignment comprises alignment of the sequenced non-host nucleic acid sequence to a reference non-host organism nucleic acid sequence over its entire length, optionally by BLAST.

Aspect 9. The method of aspect 7 or 8, wherein:
the one or more example non-host organism nucleic acid sequences comprises a plurality of example non-host organism nucleic acid sequences; and
identifying the sequenced non-host nucleic acid sequence as specific to the non-host organism further comprises determining a most likely example non-host organism nucleic acid sequence based on one or more relative mapping metrics.

Aspect 10. The method of aspect 9, wherein the relative mapping metrics include: level of sequence identity; homology and/or length of match; and specific insertions, deletions and/or single nucleotide polymorphisms with respect to the example non-host organism nucleic acid sequences.

Aspect 11. The method of aspect 9 or 10, wherein determining the most likely example non-host organism nucleic acid sequence comprises, in a case where the homology of the sequenced non-host nucleic acid sequence with the example non-host organism nucleic acid sequences is similar for a plurality of the example non-host organism nucleic acid sequences, determining the most likely example non-host organism nucleic acid sequence based on ratios of the plurality of the example non-host organism nucleic acid sequences determined as the most likely example non-host organism nucleic acid sequence from sequence alignment of others of the sequenced non-host nucleic acid sequences.

Aspect 12. The method of any of aspects 9 to 11, further comprising identifying position-specific sequence differences between the sequenced non-host nucleic acid sequences and the corresponding most likely example non-host organism nucleic acid sequence, optionally wherein the position-specific sequence differences comprise at least one of sequence polymorphisms, insertions, and deletions.

Aspect 13. The method of aspect 12, wherein the identified position-specific sequence differences are weighted using error data representing a likelihood of sequencing errors in the sequencing of the at least one sequenced non-host organism nucleic acid sequence.

Aspect 14. The method of aspect 12 or 13, further comprising calculating a frequency measure for one or more of the position-specific sequence differences representing the frequency of the position-specific sequence differences across plural of the sequenced non-host organism nucleic acid sequences in the sequencing data.

Aspect 15. The method of aspect 14, further comprising calculating a probability of the presence of a plurality of highly homologous non-host organisms based on the frequency measures, optionally further comprising calculating a relative ratio between the highly homologous non-host organisms.

Aspect 16. The method of any one of the preceding aspects, wherein the host is a mammal.

Aspect 17. The method of aspect 16, wherein the host is a human.

Aspect 18. The method of any one of the preceding aspects, wherein the non-host organism is a micro-organism and/or a parasite.

Aspect 19. The method of aspect 18, wherein the microorganism is a bacterium, a virus, a parasite, a bacteriophage, or a fungus.

Aspect 20. The method of any one of the preceding aspects, wherein the non-host organism is a pathogen.

Aspect 21. The method of aspect 20, wherein the detection and quantification of the non-host organism identifies a pathogen most likely responsible for an infection or disease in the host.

Aspect 22. The method of aspect 21, wherein the disease is a systemic infection, a local infection, a urinary tract infection, an infection of the blood, digestive tract infection, a central nervous system infection, a cardiovascular infection, an intro-abdominal infection, a urogenital tract infection, a genital tract (such as vaginal) infection, a respiratory infection and/or a skin infection.

Aspect 23. The method of any one of aspects 20 to 22, wherein the method further comprises selecting an agent suitable to treat the pathogen.

Aspect 24. The method of aspect 23, wherein the agent is an antibiotic or an antifungal.

Aspect 25. The method of any one of the preceding aspects, wherein the at least one sequenced non-host organism nucleic acid sequence is greater than 500 bp, 750 bp, or 1000 bp in length.

Aspect 26. The method of aspect 25, wherein:
a) the sequenced non-host organism nucleic acid sequence is the whole genome sequence of the non-host organism; or
b) multiple non-host organism nucleic acid sequences are sequenced to provide the whole genome sequence of the non-host organism.

Aspect 27. The method of any one of the preceding aspects, wherein the method further comprises identifying one or more antimicrobial resistance genes in the sequenced non-host organism nucleic acid sequences, optionally by sequence alignment of the sequenced non-host organism nucleic acid sequences to one or more example antimicrobial resistance gene sequences.

Aspect 28. The method of any one of the preceding aspects, further comprising identifying one or more plasmids and/or phages in the sample based on the sequencing data, optionally by comparison of the sequenced non-host organism nucleic acid sequences with one or more example plasmid and/or phage nucleic acid sequences.

Aspect 29. The method of any one of the preceding aspects, further comprising estimating a probability of relapse and/or reinfection of the host by the non-host organism based on the amount of the non-host organism.

Aspect 30. A method for detecting and quantifying the presence of a non-host organism in a host using a sample from the host, wherein the method comprises:
obtaining non-host organism nucleic acid from the sample;
sequencing at least one non-host organism nucleic acid sequence using the non-host organism nucleic acid to obtain sequencing data; and
detecting and quantifying the presence of the non-host organism using the method of any preceding claim.

Aspect 31. The method of aspect 30, wherein the detection and quantification does not require amplification of the non-host organism nucleic acid sequence.

Aspect 32. The method of aspect 30 or 31, wherein the method comprises substantial depletion of host cells from the sample obtained from the host.

Aspect 33. The method of any one of aspects 30-32, wherein the method comprises the addition of a known quantity of at least one reference non-host organism to the sample obtained from the host.

Aspect 34. The method of any one of aspects 30-33, wherein the non-host organism is cellular and the method comprises substantial lysis of the non-host organism cells.

Aspect 35. The method of aspect 34, wherein the lysis of non-host organism cells is performed by enzymatic digestion, thermal and/or physical disruption.

Aspect 36. The method of aspect 35, wherein the lysis of non-host organism cells is performed by enzymatic digestion, optionally using proteinase K, bead bashing, thermal disruption and/or sonication.

Aspect 37. The method of any one of aspects 30-36, wherein the method comprises generating a sequencing library from the non-host nucleic acid.

Aspect 38. The method of any one of aspects 30-37, wherein the sequencing is nanopore sequencing.

Aspect 39. The method of any one of aspects 30-38, wherein:
(i) detection and quantification of the non-host organism does not require amplification of the non-host nucleic acid sequence;
(ii) the method comprises substantial depletion of host cells from the sample;
(iii) the method comprises addition of a known quantity of at least one reference non-host organism to the sample;
(iv) the method comprises substantial lysis of non-host organism cells;
(v) the method comprises generating a sequencing library from the non-host nucleic acid;
(vi) the method comprises sequencing at least one non-host organism nucleic acid sequence of greater than 500 bp, 750 bp, or 1000 bp in length; and
(vii) the method comprises identifying the sequenced non-host nucleic acid sequence as specific to a particular non-host organism by alignment of the sequenced non-host nucleic acid sequence to an example non-host organism nucleic acid sequence over its entire length, optionally by BLAST.

Aspect 40. The method of any one of aspects 30-39, wherein the non-host nucleic acid is not extracted or purified from the sample prior to sequencing.

Aspect 41. The method of any one of aspects 30-40, wherein the-non-host organism is a bacterium and the method comprises sequencing non-genomic DNA of the non-host organism, optionally wherein the non-genomic DNA is a plasmid and/or bacteriophage.

Aspect 42. The method of any one of aspects 30-41, comprising sequencing of one or more antimicrobial resistance genes of the non-host organism.

Aspect 43. The method of any one of aspects 30-42, wherein the method is conducted on a saliva, blood, urine, tissue, mucus, vaginal swab, faeces, semen, spinal fluid and/or plasma sample obtained from the host.

Aspect 44. The method of any one of aspects 30-43, wherein the method is conducted on a urine sample from the host and the non-host organism is a bacterium and wherein the detection and quantification of the bacterium identifies the bacterium as a pathogen most likely responsible for a urinary tract infection.

Aspect 45. A method of treating a disease or infection associated with a pathogen in a host, wherein the method comprises detecting and quantifying the pathogen according to the method of any one of aspects 1 to 44 and administering an agent suitable to treat the pathogen, optionally wherein the agent is an antibiotic or an antifungal.

Aspect 46. A method of monitoring the effectiveness of a treatment of a disease or infection associated with a pathogen in a host, wherein the method comprises detecting and quantifying the pathogen according to the method of any one of aspects 1 to 44 and determining whether the treatment decreases the quantity of the pathogen in a sample obtained from the host.

Aspect 47. A kit comprising:
(i) a means for depleting host cells from a sample from a host;
(ii) one or more reference non-host organisms in known quantities; and
(iii) a means for generating a sequence library from non-host nucleic acids.

Aspect 48. An apparatus for detecting and quantifying the presence of a non-host organism in a host based on a sample obtained from the host, the apparatus comprising:
a determining unit configured to determine an amount of sequenced non-host organism nucleic acid sequence in the sample using sequencing data comprising at least one sequenced non-host organism nucleic acid sequence obtained from sequencing non-host organism nucleic acid from the sample; and
a quantifying unit configured to quantify an amount of the non-host organism based on the amount of the sequenced non-host organism nucleic acid sequence.

Aspect 49. A computer program comprising instructions which, when the program is executed by a computer, cause the computer to carry out the method of any of aspects 1-29, or the method of either of aspects 44 and 45 when dependent on one of aspects 1-29.

Aspect 50. A computer-readable storage medium comprising instructions which, when executed by a computer, cause the computer to carry out the method of any of aspects 1-29, or the method of either of aspects 44 and 45 when dependent on one of aspects 1-29.

### EXAMPLES

### Example 1—Method for Detecting and Quantifying the Presence of an Organism in a Urine Sample

### Materials—Equipment

MP Biomedicals MP Fastprep 24 5G machine
UVP HB-500 Minidizer Hybridisation oven
Diagenode BioRuptor Plus Sonicator
BMG Pherastar plate reader
Labnet Accublock Digital Dry Bath

### Materials—Consumables

ZymoBiomics HostZERO Microbial DNA Kit (D4310-A)
ZymoBiomics Spike in I (High Microbial Load-D6320)
1.5 mL Eppendorfs tubes
1.5 mL Diagenode Sonicating tubes
2 mL Eppendorf tubes
5 mL Eppendorf tubes
Ambion PCR Grade Water
NEB Proteinase K (20 mg/mL)
Biotium AccuBlue High Sensitivity Enhancer
Invitrogen Quant-IT PicoGreen dsDNA Reagent
NEB Lambda Phage DNA
384 well plate

### Method

### Step 1. Bacterial Pellet Collection and Host Cell Depletion

Urine samples were placed at 37° C. in a UVP HB-500 Minidizer Hybridisation oven and rotated at 12 rpm for 30 minutes. 5 mL of each urine sample was then transferred into a 5 mL Eppendorf tube and centrifuged at 21,000×g for 5 minutes. The supernatant was discarded and the pellet re-suspended in 100 μL of water.

Resuspended pellets were processed with a ZymoBiomics HostZERO Microbial DNA Kit (D4310-A) using a slightly altered manufacturer's protocol.

Namely: 500 μL of HDS was then added to the sample and subsequently rotated on a daisy wheel for 15 minutes at room temperature, centrifuged at 10,000×g for 5 minutes, supernatant discarded and the pellet re-suspended in 100 μL of MSB. 1 μL of Microbial Selection Enzyme was added, sample vortexed and incubated at 37° C. for 30 minutes followed by the addition of 20 μl Proteinase K and then re-vortexed and further incubated at 55° C. for 5 minutes. 100 μL of RNA/DNA Shield was then added, sample vortexed and subsequently incubated at room temperature for 5 minutes.

### Step 2. Bacterial Lysis and Non-Host DNA Recovery

At this stage 100 μL of water and 50 μL of ZymoBiomics Spike in I internal calibrator reference (D6320) were added along with 750 μL of ZymoBiomics Lysis Solution. Samples were vortexed, transferred to a fresh Zymo Bashingbead Lysis Tube and incubated at 95° C. for 5 minutes. The sample was transferred to a MP Biomedicals MP Fastprep 24 5G machine and the manufacturers standard E. coli programme (30 secs 6 m/s) selected and run.

Samples were then centrifuged at 15,000×g for 1 minute, 750 μL of supernatant transferred to a fresh 1.5 mL Eppendorf tube which was further centrifuged at 15,000×g for 1 minute. 300 μL aliquots of supernatant were transferred to two 1.5 mL Diagenode sonicating tubes which were then sonicated using a Diagenode BioRuptor Plus Sonicator on high power for 20 seconds. 900 μL of ZymoBiomics DNA Binding Buffer was then added to each tube and briefly vortexed. 800 μL of each sample was then loaded onto a fresh Zymo-Spin IC-Z column and centrifuged at 10,000×g for 1 minute. The flow through was discarded and the remaining sample loaded onto the Zymo-Spin IC-Z column and centrifuge at 10,000×g for 1 minute. The flow through was discarded and Zymo-Spin IC-Z column transferred to a fresh collection column. 400 μL of ZymoBiomics DNA Wash Buffer 1 was added to the Zymo-Spin IC-Z column which was centrifuged at 10,000×g for 1 minute. The flow through was then discarded and 700 μL of ZymoBiomics DNA Wash Buffer 2 was added to the Zymo-Spin IC-Z column which was centrifuged at 10,000×g for 1 minute. The flow through was once again discarded and 200 μL of ZymoBiomics DNA Wash Buffer 2 was added to the Zymo-Spin IC-Z column which was again centrifuged at 10,000×g for 1 minute. The Zymo-Spin IC-Z column was transferred to a fresh 1.5 mL Eppendorf tube and 20 μL of DNase/RNase free water was added to the Zymo-Spin IC-Z column. The Zymo-Spin IC-Z column was then incubated at room temperature for 3 minutes before centrifuging at 10,000×g for 1 minute. 20 μL flow through was captured stored at 4° C. until Nanopore sequencing libraries were made as described in Example 2.

98 μL of PCR grade water and a 2 μL aliquot of the flow through were added to a fresh 0.5 mL Eppendorf to give a 1 in 50 dilution for DNA quantification. 10 μL of the DNA sample was then added to 10 μL of quantitation mix (178 μL Ambion PCR grade water, 20 μL Biotium AccuBlue High Sensitivity Enhancer 100×, 2 μL Invitrogen Quant-IT PicoGreen dsDNA Reagent), mixed by pipetting and transferred to a fresh well of a 384 well plate. Fluorescence based quantitation of the sample was then performed on a BMG Pherastar plate reader against a pre-made log dilution series of NEB Lamdba phage DNA.

### Example 2 Library Synthesis from Sample DNA

### Materials—Equipment

Oxford Nanopore Sequencing kit SQK-LSK110
PC running Oxford Nanopore MinKNOW Sequencing Software
Oxford Nanopore MkIb MinION device and Flongle adapter
Microcentrifuge
Magnetic tube rack
Desk top daisywheel rotor

### Materials—Consumables

Oxford Nanopore Flongle R9.4 SpotOn flowcell
NEBNext FFPE DNA Repair Buffer (E6622A, Lot 10060502)
NEBNext FFPE DNA Repair Mix (NEB M6630S)
NEBNext Ultra II End Prep Reaction Buffer (NEB E7647A)
NEBNext Ultra II End Prep Enzyme Mix (E7646A, Lot 10094514)
NEBNext Quick T4 DNA Ligase (E6057A, lot 10054713)
Ambion PCR grade Water
Ambion AMPure XP beads
70% EtOH
Extracted Urine DNA sample prepared according to Example 1

### Method

DNA samples obtained from urine using the method described in Example 1 were processed into Oxford Nanopore compatible Sequencing libraries using a modified Oxford Nanopore Sequencing SQK-LSK110 kit protocol for Flongle.

20 μL of sample DNA was added to a reaction mixture comprising: 1.75 μL NEBNext FFPE DNA Repair Buffer (E6622A), 1.0 μL NEBNext FFPE DNA Repair Mix (NEB M6630S), 1.75 μL NEBNext Ultra II End Prep Reaction Buffer (NEB E7647A), 1.5 μL NEBNext Ultra II End Prep Enzyme Mix (E7646A, Lot 10094514), 4 μL water. The solution was then mixed before incubating at 20° C. for 5 minutes then 65° C. for 5 minutes. The sample was then held at 4° C.

70 μL of water was then added to sample along with 100 μL Ambion AMPure XP beads. The sample was then vortexed to suspend the beads and transferred to a 2.0 mL Eppendorf tube before incubating at room temperature on desktop daisywheel rotor for 5 minutes.

The reaction was briefly collected by centrifuge (10,000×g for 10 seconds) and then placed on a magnetic rack for 2 minutes to collect beads. The supernatant was carefully discarded and the magnetic pellet washed with 250 μL of 70% EtOH and vortexed to mix. The pellet was briefly collected by centrifuge (10,000×g for 10 seconds) and then placed back on a magnetic rack for 2 minutes to recollect the beads. The supernatant was again carefully discarded and the magnetic pellet washed with a further 250 μL of 70% EtOH and vortexed to mix. The pellet was briefly collected by centrifuge (10,000×g for 10 seconds) and then placed back on a magnetic rack for 2 minutes to recollect the beads then the supernatant discarded. The beads were re-suspended in 12 μL clean water and left at room temperature for 2 minutes then re-pelleted on the magnet rack and 12 μL eluate transferred to a fresh 2 mL LoBind tube.

12 μL eluate was added to a fresh 2.0 ml Eppendorf tube with 6 μL LNB Buffer, 2 L NEBNext Quick T4 DNA Ligase (E6057A) and 2 μL Adapter Mix (AMX F). The reaction mixture was incubated at 20° C. for 10 minutes on a desktop daisy wheel. Following the incubation, 75 μL of water and 100 μL Ambion AMPure XP beads were added to the reaction mixture mixed by vortexing and then placed on a desktop daisy wheel rotor at room temperature for 5 minutes.

Following the incubation, the mixture was collected in a centrifuge (10,000×g for 10 seconds) and then placed on a magnetic rack for 2 minutes to collect beads then the supernatant discarded. 125 μL Oxford Nanopore Long Fragment Buffer (LFB) was then added to the re-suspend pellet, spun in a centrifuge (10,000×g for 10 seconds) and then placed on a magnetic rack for 2 minutes and any residual supernatant discarded. Beads were then removed from the magnetic rack and re-suspended in 10 μL Elution Buffer (EB) and incubated at 37° C. for 5 minutes. Finally the mixture was collected in a centrifuge (10,000×g for 10 seconds), placed on a magnetic rack for 2 minutes to collect beads and a 10 μL eluate containing the final DNA Sample Library was transferred to a fresh 1.5 mL LoBind tube.

Flongle Flow Cell priming was set up in accordance with the manufacturing instructions and guidelines. In a 1.5 mL Eppendorf tube, 3 μL Flush Tether (FLT) and 117 μL Flush Buffer (FB) were mixed. 100 μL of the FLT and FB mixture was slowly added into Spot ON port of a fresh Flongle R9.4 SpotOn flowcell and the introduction of air bubbles was avoided.

A reaction mixture comprising 13.5 μL of Sequencing buffer SBII, 11 μL of Oxford Nanopore Loading Beads LBII and 10 μL of DNA Sample Library was mixed in a fresh 1.5 mL Eppendorf tube. 30 μL of the reaction mixture was gently loaded onto a fresh Flongle flow cell using SpotON port and sequencing run using manufacturers default settings.

Sequencing data was collected overnight, typically for 14 hours. Resulting Fastq datasets were uploaded to a bespoke cloud-based analysis suite for processing. Individual reads were filtered for quality, length and chimerism then aligned (BLAST (Altschul et al: Basic local alignment search tool. J Mol Biol 1990, 215(3):403-410)) to a curated database of unique species-level bacterial, yeast, archaea or host references including a separately curated non-redundant plasmid and phage dataset. Alignments were secondarily filtered for various metrics to remove low quality mappings and noise. Per-species post-filtered mapping data was used to estimate sample input cell values by reference to mapping data for calibrator spike species. Results from individual samples were collated into ‘heat-map’ diagrams displaying estimated input cell numbers per species (per mL for urine input, per swab for vaginal) and displayed using a quantity-specific colourised scale. Data points resulting from less than 10 unique high-quality read mappings or equating to <1,000 cells/mL were excluded.

Consensus assemblies were created and exported for species of particular interest where aligned read mapping exceeded 10× coverage genome-wide. SNP comparison tools (Treangen et al: The Harvest suite for rapid core-genome alignment and visualization of thousands of intraspecific microbial genomes. Genome Biol 2014, 15(11):524) [34]) were used to compare multiple strains of the same species. Core genome phylogenies were output in Newick format and used to produce cladograms (Letunic and Bork P: Interactive Tree Of Life (iTOL) v5: an online tool for phylogenetic tree display and annotation. Nucleic Acids Res 2021, 49(W1):W293-W296) predicting sub-strain homologies or serotypes where applicable.

### Example 3—Establishing Accuracy of Quantitation

### Materials—Consumables

Zymo mock microbial community D6300
PCR grade water
Zymo RNA/DNA shield

A commercial mix of microorganisms (Zymo D6300) was used to assess the accuracy of the method set out in Example 1. The commercial mix of microorganisms contained:
3 gram negative bacteria species—Escherichia coli, Salmonella enterica, Pseudomonas aeruginosa;
5 gram positive bacteria species—Bacillus subtilis, Enterococcus faecalis, Lactobacillus fermentum, Listeria monocytogenes and Staphylococcus aureus; and
2 fungi species—Cryptococcus neoformans and Saccharomyces cerevisiae.

Each known species within the mixture was provided at a predicted concentration of cells/mL allowing both qualitative and quantitative assessment of the accuracy of the method. The manufacturer's supplied estimation of input cell numbers for Zymo product D6300 was adopted with all data given as relative to the published manufacturer's estimates.

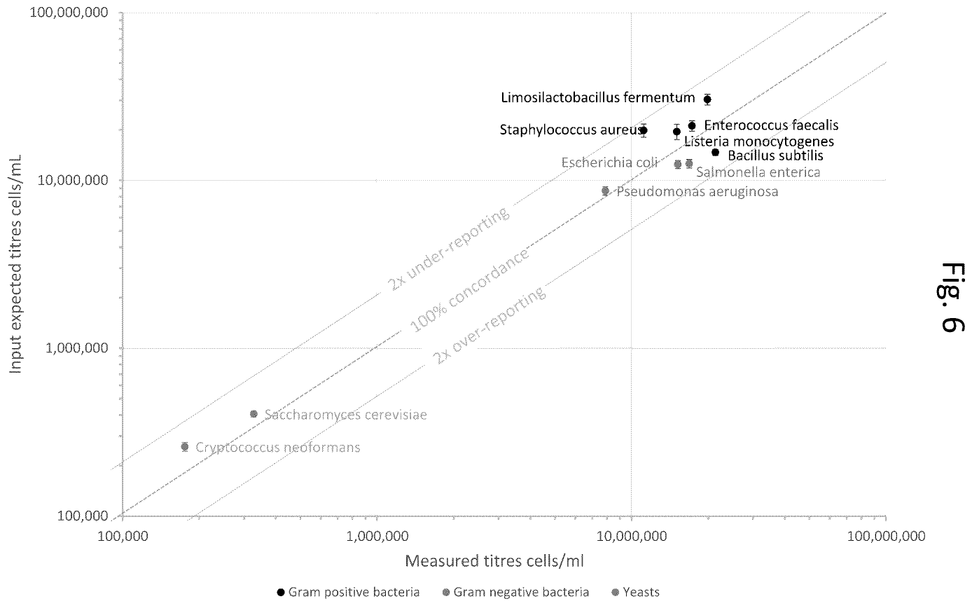

Zymo D6300 was diluted 1 in 2.5 in PCR grade water. 10 μL of this dilution was added to 100 μL of Zymo RNA/DNA shield in a fresh Eppendorf and processed according to Example method 1, beginning at Step 2. Bacterial lysis and non-Host DNA recovery. This experiment was independently repeated four times to assess accuracy and reproducibility. The results are listed in Table 1 and illustrated in FIG. 5. The average cell count and standard deviation for each species is listed in Table 2.

As demonstrated in Tables 1 and 2, and FIG. 5, the method was capable of simultaneously detecting and measuring cell counts for all ten microorganisms from the mixed population comprising both gram-negative and gram-positive bacteria as well as multiple fungal species. For the replicates of the 1 in 2.5 dilution there was an average percentage error of 19.5% (error range 8.8% to 35.7%) in the observed cell numbers compared to the manufacturer's supplied cell counts demonstrating a very close correlation. This consistency between independent replicates represents less than 0.5× fold deviation from the expected cell number.

TABLE 1

| | Expected | | | | |
|---|---|---|---|---|---|
| | Cell | Estimated Cell Number | | | |
| Organism | Number | Repeat 1 | Repeat 2 | Repeat 3 | Repeat 4 |
| Pseudomonas | 8,669,364 | 11,289,385 | 9,973,952 | 9,192,165 | 9,920,520 |
| aeruginosa | | | | | |
| Escherichia | 12,450,784 | 14,277,614 | 11,285,666 | 17,772,847 | 10,871,486 |
| coli | | | | | |
| Salmonella | 12,577,531 | 19,465,469 | 16,430,102 | 16,729,165 | 15,630,297 |
| enterica | | | | | |
| Lactobacillus | 30,385,893 | 23,115,326 | 27,854,047 | 22,982,834 | 25,256,467 |
| fermentum | | | | | |
| Enterococcus | 21,134,354 | 25,438,663 | 23,944,707 | 26,056,428 | 21,198,624 |
| faecalis | | | | | |
| Staphylococcus | 19,878,446 | 14,023,190 | 13,462,430 | 15,232,358 | 12,635,597 |
| aureus | | | | | |
| Listeria | 19,527,957 | 18,521,816 | 17,924,210 | 18,166,334 | 15,890,024 |
| monocytogenes | | | | | |
| Bacillus | 14,710,384 | 21,162,750 | 19,086,924 | 17,901,927 | 14,996,066 |
| subtilis | | | | | |
| Saccharomyces | 406,132 | 390,080 | 413,970 | 557,166 | 407,715 |
| cerevisiae | | | | | |
| Cryptococcus | 259,154 | 251,549 | 364,094 | 392,007 | 318,426 |
| neoformans | | | | | |

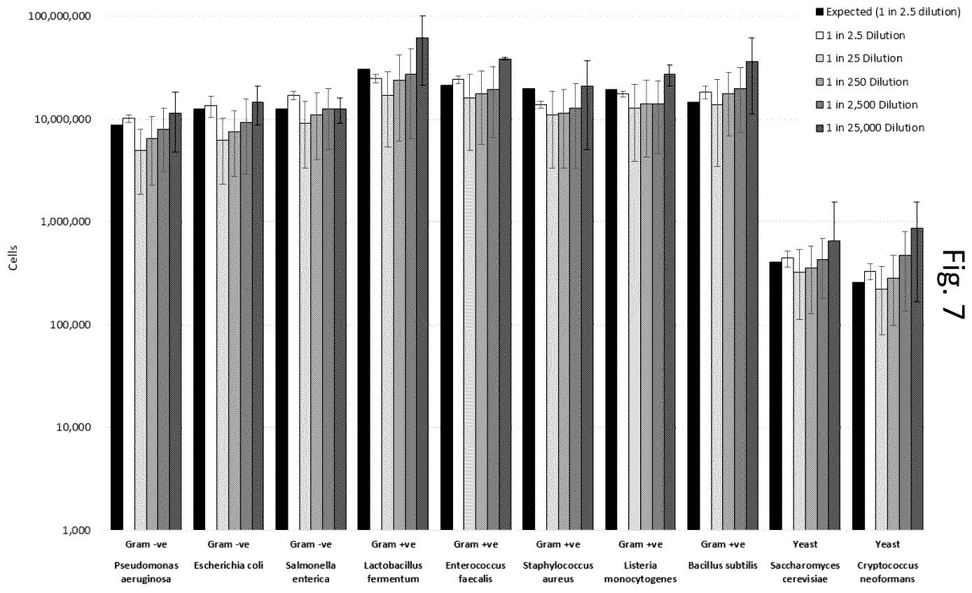

As a continuation of the above analysis, a further 6 repeats were conducted on the Zymo product D6300. Again the method was able to qualitatively identify all ten species present (FIG. 6). Across the six replicates, 0.04% ( 542/1,251,167) of reads mapped to species other than those expected in the input. This maximum off-target or misidentification rate reduced to 0.012% when excluding species identified from a single mapped read and was eliminated completely when only including species identified from ten or more mapped reads. Further, concordance of predicted absolute cell numbers against expected cell input values across all species suggests our method represents a highly-quantitative assay (FIG. 6). Standard deviation values from the six independent replicates indicates the high reproducibility of the method. In addition, there was little obvious deviation associated with organism or gram-stain type indicating good uniformity of cell lysis and DNA isolation across species.

TABLE 2

| | Expected | Average | Standard |
|---|---|---|---|
| Organism | Cell Number | Cell Number | deviation (σ) |
| Pseudomonas aeruginosa | 8,669,364 | 10,094,006 | 873,071 |
| Escherichia coli | 12,450,784 | 13,551,903 | 3,197,055 |
| Salmonella enterica | 12,577,531 | 17,063,758 | 1,666,983 |
| Lactobacillus fermentum | 30,385,893 | 24,802,169 | 2,285,882 |
| Enterococcus faecalis | 21,134,354 | 24,159,606 | 2,163,910 |
| Staphylococcus aureus | 19,878,446 | 13,838,394 | 1,090,161 |
| Listeria monocytogenes | 19,527,957 | 17,625,596 | 1,182,792 |
| Bacillus subtilis | 14,710,384 | 18,286,917 | 2,574,771 |
| Saccharomyces cerevisiae | 406,132 | 442,233 | 77,287 |
| Cryptococcus neoformans | 259,154 | 331,519 | 61,337 |

### Example 4—Establishing Sensitivity of Quantitation

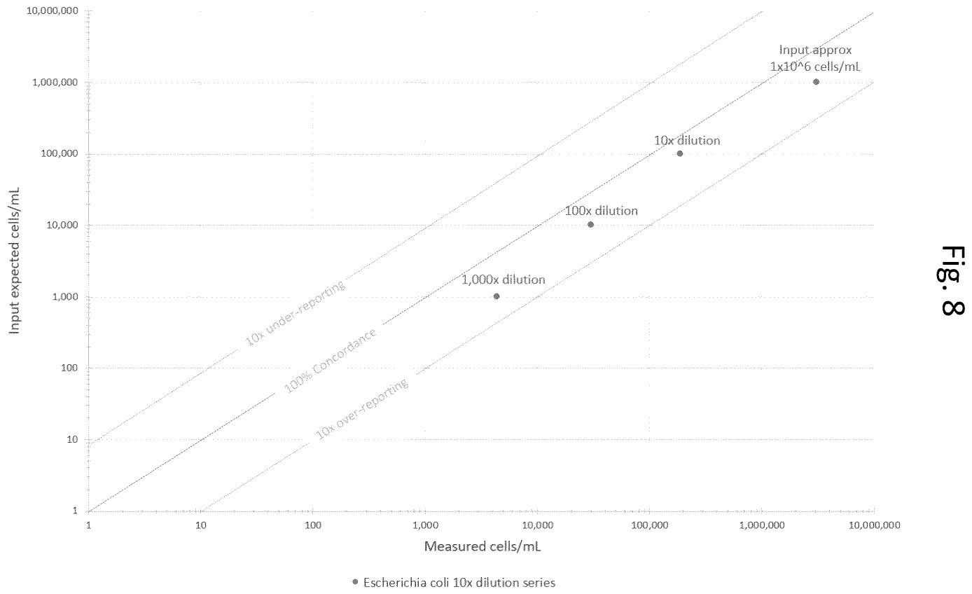

To assess the sensitivity and robustness of the method illustrated in Example 1, a commercial mix of microorganisms (Zymo D6300) was again used but across a range of dilutions. Zymo D6300 was diluted at 1 in 2.5, 1 in 25, 1 in 250, 1 in 2,500, and 1 in 25,000 in PCR grade water. 10 μL of each dilution was added to 100 μL of Zymo RNA/DNA shield in a fresh Eppendorf and processed according to the method described in Example 1, beginning at Step 2. Bacterial lysis and non-Host DNA recovery. Each dilution was independently repeated four times. The average results for each dilution are listed in Table 3 and illustrated in FIG. 7.

Table 3 demonstrates the ability of the method to detect and quantify cell numbers for the mixed organisms across a five-log dilution range in the sample input. The results demonstrate that the sensitivity of the method enables all ten organisms to be detected at a 100 to 1,000 cell level in the sample input. Although there was a trend for the standard deviations to increase as the amount of cells decreased in the sample input, the method nonetheless provided reliable and accurate cell counts at the low cell level in the sample input. FIG. 7 illustrates the results of multiplying the average input cell estimates of each dilution series from Table 2 by the input dilution factor to provide an estimate of cell numbers in the undiluted sample. This allows the quantitative nature of each dilution log to be clearly compared across the entire series and reinforces that reproducibility and accuracy continue across the full range of log dilutions.

### Example 5—Establishing Sensitivity of Quantitation

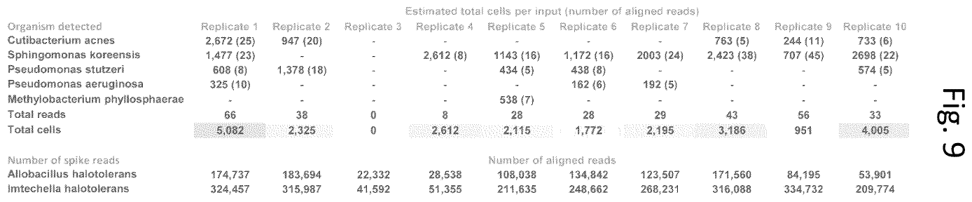

In a further analysis of the sensitivity of the method illustrated in Example 1, a 10× log dilution series of an approximate 1×10⁶ cell/mL monoclonal Escherichia coli culture (FIG. 8) were measured. Escherichia coli monoclonal cultures were grown, diluted and adjusted to approximately 1×10⁶ cells/mL using OD measurements. Titres were confirmed using orthogonal measures such as cfu counts on culture plates of log series dilutions, expected vs observed yields from DNA extractions and microbial cell counter analysis (QUANTOM Tx, LOGOS Bio).

Accuracy of measured values remained high up to approximately 1×10³ cell inputs per species, indicating that the method is capable of accurate and sensitive identification of species in concentrations below 1×10⁵ cell/mL, the concentration reporting threshold employed by NHS culture methods (HE: SMI B41: investigation of urine. Information on UK standards for microbiology investigations of urine. Public Health England 2018). Highly sensitive microbiome profiling methodologies are prone to low-level background contaminants that can arise from sample handling, plastic-ware or commercial reagents. To establish the baseline process specific signal of the method illustrated in Example 1, ten independent replicates of fresh, sterile ultrapure water were analysed. The quantitative analysis showed that 9/10 samples returned a low diversity collection of species (>5 mapped reads/species) primarily consisting of Sphingomonas koreensis, Cutibacterium acnes, Pseudomonas stutzeri and Pseudomonas aeruginosa (FIG. 9). Our analysis therefore suggests the common contaminates of the method are readily identifiable bacterial species that contribute less than 5,000 estimated cells per processed input sample.

TABLE 3
Average predicted input cells are given from three independent replicates
run for each dilution series (+/−1 standard deviation).

| | Expected | Estimated Cell Number | | | | |
|---|---|---|---|---|---|---|
| | Cell Number | 1 in 2.5 | 1 in 25 | 1 in 250 | 1 in 2,500 | 1 in 25,000 |
| Organism | (1 in 2.5 dilution) | dilution | dilution | dilution | dilution | dilution |
| Pseudomonas aeruginosa | 8,669,364 | 10,094,006 | 615,817 | 79,929 | 9,880 | 1,150 |
| | | (+/−873,071) | (+/−162,245) | (+/−23,559) | (+/−2,216) | (+/−675) |
| Escherichia coli | 12,450,784 | 13,551,903 | 783,003 | 93,059 | 11,586 | 1,471 |
| | | (+/−3,197,055) | (+/−205,389) | (+/−24,895) | (+/−4,297) | (+/−604) |
| Salmonella enterica | 12,577,531 | 17,063,758 | 1,129,875 | 138,212 | 15,570 | 1,247 |
| | | (+/−1,666,983) | (+/−302,311) | (+/−39,113) | (+/−3,045) | (+/−341) |
| Lactobacillus fermentum | 30,385,893 | 24,802,169 | 2,134,842 | 300,649 | 33,973 | 6,158 |
| | | (+/−2,285,882) | (+/−792.927) | (+/−137,216) | (+/−16,177) | (+/−4,012) |
| Enterococcus faecalis | 21,134,354 | 24,159,606 | 2,000,542 | 220,250 | 24,411 | 3,862 |
| | | (+/−2,163,910) | (+/−747,844) | (+/−78,944) | (+/−8,089) | (+/−116) |
| Staphylococcus aureus | 19,878,446 | 13,838,394 | 1,364,517 | 141,896 | 15,904 | 2,094 |
| | | (+/−1,090,161) | (+/−522,960) | (+/−56,241) | (+/−7,050) | (+/−1,590) |
| Listeria monocytogenes | 19,527,957 | 17,625,596 | 1,591,226 | 176,097 | 17,584 | 2,728 |
| | | (+/−1,182,792) | (+/−609,102) | (+/−68,652) | (+/−6,202) | (+/−630) |
| Bacillus subtilis | 14,710,384 | 18,286,917 | 1,737,693 | 218,926 | 24,464 | 3,624 |
| | | (+/−2,574,771) | (+/−801,398) | (+/−49,780) | (+/−6,324) | (+/−2,624) |
| Saccharomyces cerevisiae | 406,132 | 442,233 | 40,741 | 4,458 | 539 | 65 |
| | | (+/−77,287) | (+/−13,152) | (+/−1,297) | (+/−89) | (+/−92) |
| Cryptococcus neoformans | 259,154 | 331,519 | 27,870 | 3,552 | 585 | 86 |
| | | (+/−61,337) | (+/−8,257) | (+/−3,552) | (+/−238) | (+/−69) |

### Example 6—Comparison of 16s rRNA Profiling Methods with Zymo D6300

Comparative experiments were conducted between the method set out in Example 1 and two known 16s rRNA profiling methods: Illumina 16s rRNA profiling and Oxford Nanopore 16s rRNA profiling. Initial experiments were conducted using Zymo D6300, a commercially available mix of ten microorganisms at known concentrations, to give an input reagent with known composition.

### Materials—Consumables

Zymo mock microbial community D6300
PCR grade water
Zymo RNA/DNA shield
GenXPro 16S rRNA-Seq Metagenomic Library Preparation Kit
Oxford Nanopore sequencing kit SQK-RAB204 and Flongle R9.4 flowcell

### Input Preparation

Zymo D6300 was diluted at 1:2.5 in PCR grade water. 10 μL aliquot of this mix was added to 100 μL of Zymo RNA/DNA shield in a fresh Eppendorf and used as inputs into the following workflows.

Illumina 16s rRNA Method

DNA was extracted according to the method of Example 1, beginning at Step 2. Bacterial lysis and non-Host DNA recovery but without the addition of ZymoBiomics Spike I internal calibrator reference (D6320). Extracted DNA was used as input into GenXPro 16S rRNA-Seq Metagenomic Library Preparation Kit. Prepared sequencing libraries were sequenced using Illumina MiSeq platform at 300 bp PE protocol. Resultant data was analysed using the Qiime 2 package.

Nanopore 16s rRNA Method

DNA was extracted according to the method of Example 1, beginning at Step 2. Bacterial lysis and non-Host DNA recovery but without the addition of the ZymoBiomics Spike I internal calibrator reference (D6320). Extracted DNA was used as input into Oxford Nanopore sequencing kit SQK-RAB204, run on an Oxford Nanopore Flongle R9.4 flow cell and resultant sequencing data analysed using Nanopore Epi2Me 16s rRNA cloud workflow.

TABLE 4
Expected microorganism identification and relative quantity
of Zymo D6300 based on 16s rRNA sequencing using Illumina
and Oxford Nanopore 16s rRNA sequencing methods.

| | Expected % Composition of Zymo D6300 | | |
|---|---|---|---|
| | based on 16s rRNA copy number | | |
| | Manufacturer | | Oxford |
| | predicted % | Illumina | Nanopore |
| Organism | by 16sRNA | Method | Method |
| Bacillus subtilis | 17.4 | 19.3* | 13.7 |
| Enterococcus faecalis | 9.9 | 14.7* | 12.4 |
| Lactobacillus fermentum | 18.4 | 20.2 | 11.0 |
| Listeria monocytogenes | 14.1 | 12.3* | None |
| | | | Detected |
| Staphylococcus aureus | 15.5 | 12.0* | 11.6 |
| Escherichia coli | 10.1 | 15.3* | None |
| | | | Detected |
| Pseudomonas aeruginosa | 4.2 | 5.7* | 2.5 |
| Salmonella enterica | 10.4 | None | 22.0 |
| | | Detected | |
| Cryptococcus neoformans | NA⁺ | NA⁺ | NA⁺ |
| Saccharomyces cerevisiae | NA⁺ | NA⁺ | NA⁺ |

⁺16s rRNA based methods cannot identify fungal species Crytococcus neoformans and Saccharomyces cerevisiae as fungi do not contain 16s rRNA genes.
*Organism detected to genus level only.

Table 4 shows the results of applying two commercial 16s rRNA profiling methodologies using the same commercial mix of microorganisms (Zymo D6300) used in Example 3 as the input sample. Both methods were incapable of providing estimated absolute cell numbers as neither contained an internal calibrator reference, hence all data is shown as percentage (%) composition (based on 16s RNA sequence read numbers) of identified organisms. This allows direct comparison with the expected 16s rRNA predicted percentage composition of the Zymo D6300 microorganism mixture from the manufacturers details (available at https://files.zymoresearch.com/protocols/_d6300_zymobiomics_microbial_community_standard.pdf).

Table 4 shows that the Illumina 16s rRNA method was unable to detect the majority of organisms at species level resolution, giving only genus level identifications for six out of the ten organisms present. This method also failed to detect Salmonella enterica. Only one organism, Lactobacillus fermentum, was correctly identified as being present at species level by the Illumina 16s rRNA method. Similarly, the Oxford Nanopore 16s rRNA method was unable to identify either Listeria monocytogenes or Escherichia coli and only provided correct species level identifications for six of the other organisms present.

Quantitative analysis showed significant variation between the measured % composition for each technique against the expected values provided by the manufacturer. Additionally, neither method was able to provide estimated absolute cell numbers or identify fungal species. There is also no normalisation in either 16sRNA method to account for differing 16sRNA gene copy numbers between bacterial species. The data from Examples 3 and 4 (Tables 1, 2 and 3, FIGS. 5 and 7) highlights the much tighter correlation of quantitation (including absolute cell numbers) with the manufacturer's expected values that is possible when using the method described herein.

Comparison of the data from Examples 3 and 4 also shows that the method described herein was the only protocol tested capable of identifying all species of microorganism present in the commercial mix and providing an estimate of the cell count in the sample.

### Example 7—Comparison of Performance of the Method of Example 1 and Existing 16s rRNA Profiling Methods with Donor Urine Samples

Comparative experiments were conducted using the 16s rRNA methods described in Example 6, Illumina 16s rRNA profiling and Oxford Nanopore 16s rRNA profiling, with the method of Example 1.

Two symptomatic UTI patients and two asymptomatic healthy controls provided urine samples for use as the input samples in the methods described below. The organism composition of the samples was undefined and unknown.

### Input Preparation

5 mL aliquots of urine samples from two symptomatic donors with suspected urinary tract infections and two asymptomatic healthy controls were used as sample inputs in the following methods.

### Method of Example 1

DNA was extracted, sequenced and analysed according to the method of Example 1.

Illumina 16s rRNA Method

DNA was extracted according to the method of Example 1, beginning at Step 2. Bacterial lysis and non-Host DNA recovery and without the addition of ZymoBiomics Spike I internal calibrator reference (D6320). Extracted DNA was used as input into GenXPro 16S rRNA-Seq Metagenomic Library Preparation Kit. Prepared sequencing libraries were sequenced using Illumina MiSeq platform at 300 bp PE protocol. Resultant data was analysed using the Qiime 2 package.

Nanopore 16s rRNA Method

DNA was extracted according to the method of Example 1, beginning at Step 2. Bacterial lysis and non-Host DNA recovery and without the addition of ZymoBiomics Spike I internal calibrator reference (D6320). Extracted DNA was used as input into Oxford Nanopore sequencing kit SQK-RAB204, run on an Oxford Nanopore Flongle R9.4 flow cell and resultant sequencing data analysed using Nanopore Epi2Me 16s rRNA cloud workflow.

TABLE 5
Symptomatic UTI Patient 1 (Donor CP019_18).

| | Method | | | | | | |
|---|---|---|---|---|---|---|---|
| | Illumina 16s rRNA | | Oxford Nanopore 16s rRNA | | Method of Example 1 | | |
| Organism | Identified | Predicted % | Identified | Predicted % | Identified | Predicted % | Cells/mL |
| Enterococcus faecalis | Y⁺ | 74.1 | Y | 79.2 | Y | 86.0 | 2,293,652 |
| Lactobacillus crispatus | Y⁺ | 18.7 | Y | 16.9 | Y | 8.1 | 246,124 |
| Peptoniphilus harei | Y⁺ | 0.8 | Y | 0.1 | Y | 0.8 | 25,515 |
| Streptococcus anginosus | Y | 0.7 | Y | 1.9 | Y | 0.1 | 1,733 |
| Finegoldia magna | Y⁺ | 1.2 | N | — | Y | 1.9 | 34,694 |
| Dialister micraerophilus | Y⁺ | 0.5 | Y | 0.6 | N | — | — |
| Staphylococcus epidermis | N | — | Y | 0.2 | Y | 0.2 | 6,213 |
| Prevotella disiens | Y | 0.5 | N | — | N | — | — |
| Dialister propionicifaciens | N | — | Y | 0.5 | N | — | — |
| Enterococcus rivorum | N | — | Y | 0.4 | N | — | — |
| Streptococcus periodonticum | N | — | N | — | Y | 0.9 | 35,351 |
| Enterococcus avium | N | — | N | — | Y | 1.4 | 28,223 |
| Prevotella intermedia | N | — | N | — | Y | 0.2 | 18,365 |
| Corynebacterium tuberculostearicum | N | — | N | — | Y | 0.5 | 15,185 |

⁺indicates identification of organism to genus level only.

TABLE 6
Symptomatic UTI Patient 2 (Donor FC015_01).

| | Method | | | | | | |
|---|---|---|---|---|---|---|---|
| | Illumina 16s rRNA | | Oxford Nanopore 16s rRNA | | Method of Example 1 | | |
| Organism | Identified | Predicted % | Identified | Predicted % | Identified | Predicted % | Cells/mL |
| Oligella urethralis | Y | 6.4 | Y | 10.0 | Y | 52.5 | 1,029,809 |
| Alloscardovia omnicolens | Y | 2.0 | Y | 0.6 | Y | 18.2 | 493,800 |
| Aerococcus urinae | Y | 8.1 | Y | 13.1 | Y | 14.5 | 368,825 |
| Peptoniphilus harei | Y⁺ | 5.8 | Y | 9.6 | Y | 8.5 | 237,039 |
| Campylobacter ureolyticus | Y | 6.5 | Y | 23.0 | Y | 6.4 | 161,952 |
| Prevotella timonensis | Y | 31.6 | Y | 8.2 | N | — | — |
| Dialister propionicifaciens | Y⁺ | 3.4 | Y | 18.1 | N | — | — |
| Nosocomiicoccus ampullae | Y | 5.4 | N | — | N | — | — |
| Ezakiella | Y⁺ | 2.5 | N | — | N | — | — |
| Bacteriodes coagulans | N | — | Y | 6.8 | N | — | — |
| Anaerococcus degeneri | N | — | Y | 3.1 | N | — | — |
| Actinotignum schaalii | N | — | N | — | Y | 16.9 | 369,811 |
| Corynebacterium urealyticum | N | — | N | — | Y | 13.8 | 295,845 |
| Bifidobacterium longum | N | — | N | — | Y | 5.6 | 109,624 |
| Prevotella jejuni | N | — | N | — | Y | 4.8 | 135,849 |

⁺indicates identification of organism to genus level only.

TABLE 7
Asymptomatic Healthy Control 1 (Donor CP022_04).

| | Method | | | | | | |
|---|---|---|---|---|---|---|---|
| | Illumina 16s rRNA | | Oxford Nanopore 16s rRNA | | Method of Example 1 | | |
| Organism | Identified | Predicted % | Identified | Predicted % | Identified | Predicted % | Cells/mL |
| Lactobacillus iners | Y | 98.7 | Y | 98.1 | Y | 95.7 | 1,999,126 |
| Lactobacillus vaginalis | Y⁺ | 0.7 | Y | 0.6 | Y | 2.1 | 37,948 |
| Gardnerella vaginalis | Y⁺ | 0.1 | N | — | Y | 0.7 | 11,910 |
| Streptococcus agalactiae | Y | 0.01 | N | — | Y | 0.1 | 1,549 |
| Lactobacillus reuteri | Y | 0.4 | N | — | N | — | — |
| Lactobacillus gasseri | N | — | Y | 0.8 | N | — | — |
| Aerococcus christensenii | N | — | N | — | Y | 0.4 | 7,999 |
| Mageeibacillus indolicus | N | — | N | — | Y | 0.3 | 5,772 |
| Lactobacillus kefiranofaciens | N | — | N | — | Y | 0.3 | 4,464 |

⁺indicates identification of organism to genus level only.

TABLE 8
Asymptomatic Healthy Control 2 (Donor CP024_04).

| | Method | | | | | | |
|---|---|---|---|---|---|---|---|
| | Illumina 16s rRNA | | Oxford Nanopore 16s rRNA | | Method of Example 1 | | |
| Organism | Identified | Predicted % | Identified | Predicted % | Identified | Predicted % | Cells/mL |
| Lactobacillus iners | Y | 61.8 | Y | 71.8 | Y | 44.4 | 363,544 |
| Lactobacillus crispatus | Y⁺ | 32.5 | Y | 24.0 | Y | 31.0 | 159,445 |
| Lactobacillus jensenii | Y | 2.0 | Y | 2.1 | Y | 1.4 | 10,197 |
| Aerococcus christensenii | Y | 0.2 | Y | 0.1 | Y | 0.6 | 3,986 |
| Lactobacillus gasseri | Y | 0.5 | Y | 1.0 | Y | 0.2 | 1,157 |
| Gardnerella vaginalis | Y⁺ | 1.8 | N | — | Y | 20.8 | 145,701 |
| Lactobacillus vaginalis | N | — | Y | 0.6 | Y | 0.5 | 3,097 |
| Lactobacillus amylovorus | N | — | N | — | Y | 0.4 | 2,156 |

⁺indicates identification of organism to genus level only.

Tables 5, 6, 7, and 8 display the estimated composition of microorganisms in donor samples from two symptomatic UTI patients and two asymptomatic healthy controls assayed, in parallel, using Illumina and Oxford Nanopore 16s rRNA methods compared to the method of Example 1. Predicted percentage composition values are given for each identified organism for the 16s rRNA techniques based on the number of sequencing reads recovered for each organism. The calculated cell numbers of the samples are given for the results of the method of Example 1, these are also further converted to percentage composition values to allow direct comparison across the three techniques. Only organisms predicted to be present at >0.3% in the total composition are shown, except where the same organism was detected by more than one technique.

There was some agreement in the qualitative identification of species identified as highly prevalent in the four samples across the three techniques. The calculated percentage cell composition for each of the identified species though varied between the methods, for example, Campylobacter ureolyticus in the Healthy Control 1 sample (Table 6) and Gardnerella vaginalis in the Symptomatic UTI Patient 2 sample (Table 8). Only the method of Example 1 was able to provide absolute cell numbers for the original input sample, with important consequences for diagnostic utility. A UTI infection is typically diagnosed by the presence of a known associated organism at an amount of 10⁵ CFU/ml (Kass EH 1962 Ann Intern Med Vol. pp. 46-53) which can be correlated from the amount in cells/mL and confirmed with traditional plating and culturing techniques. The lack of absolute quantitative data with the 16sRNA techniques limits their clinical applications for diagnosing infections such as UTIs.

As shown in Table 5 (Enterococcus faecalis) and Table 6 (Oligella urethralis), the method described herein is able to identify bacterial species most likely responsible for UTI infections based on absolute cell numbers and thus enables an informed treatment plan to be formulated.

Table 6 also demonstrates the challenges in using relative quantitative data (percentage) in understanding the composition of an infection and identifying the most likely organism responsible for an infection. During an infection it is common for increased proliferation to occur amongst bacterial and fungal species and the microenvironment changes through the infection, resulting in an overall increase in bacteria and fungi numbers as seen in Table 6 for all three techniques for bacterial species. This increase across the microorganism species can hide or dilute the increase of the microorganism most likely responsible for the initial infection. In contrast, the method described herein enables determination of absolute cell number which allows for the bacterial and fungal species of the microenvironment to be interrogated at a greater detail to detailed understanding of the composition of an infection and identifying the most likely organism responsible for an infection.

The method of Example 1, as illustrated above (see Example 6), allows for more accurate quantification of organisms present in an input sample and thus the results obtained for patient samples according to the method are expected to be more representative of the actual quantities of organisms in the samples compared to the other methods compared. The method of Example 1 also did not require PCR amplification of the extracted DNA prior to sequencing. This eliminates the requirement for assumptions on universal oligonucleotide primer annealing sites to be made. The method illustrated in Example 1 (in contrast to the compared methods) was also able to identify non-bacterial organisms such as fungi, viruses and/or bacteriophages, as illustrated in Table 5 as it does not require primers designed against the 16s rRNA gene.

The method illustrated in Example 1 also allowed resolution of species identification beyond the genus level. The whole genome sequencing data provided allowed genome wide variations to be analysed and organism identity to be established at a higher resolution; down to the species, strain and even sub-strain level such as the Escherichia-Shigella subgroup or Lactobacilli.

The composition and quality of reference databases is an important feature for any assay that uses a matching algorithm. The lack of an appropriate reference can result in failure to identify a component organism from a mixed sequencing dataset. Absence of a signal for organisms robustly identified by two out of three of the methods, Gardnerella vaginalis (Tables 7 and 8), Streptococcus agalactiae (Table 7), Finegoldia magna (Table 5), suggest either a lack of a suitable reference in the Oxford Nanopore method or a PCR based amplification issue. The method illustrated in Example 1 also allows for inbuilt bioinformatic tools to perform secondary analysis of sequencing reads that remain unmatched to any references in the local database. This analysis allows the software to flag and identify additional references that may be required to be added to the local reference database. Species may be misidentified in some known methods due to incomplete databases lacking references organisms or technical limitations of the methods. As an example, the claimed method allowed identification of Prevotella jejuni with a high level of confidence (Table 6). In contrast, both 16s rRNA methods identified Prevotella timonensis in closely related species. References for both species are contained in the database of Example 7, suggesting that the identification of Prevotella jejuni was more likely. Speculatively, this identification may not have been possible in the 16s rRNA methods due to a lack of a reference for this species in the databases.

### Example 8. Organism Identification Beyond the Genome

Further experiments were conducted to determine if the shotgun sequencing of long fragments of non-host DNA originating from the sample obtained according to Example 1 enabled a high resolution of sequence identification. In particular the ability to co-sequence and profile of plasmid cohorts and phage/virus cohorts, identify antimicrobial resistance sequences present in genomes or plasmids and identify genome wide SNP patterns for a mono-cultured or heavily dominant organism were analysed.

Table 9. provides an example comparison of four different urine samples taken from unrelated donors analysed using the method of Example 1. All four samples were dominated by Lactobacillus crispatus at predicted cell counts 60× to 300× more prevalent than the next most prolific species. Lactobacillus crispatus therefore accounted for the vast majority of sequenced bacterial material present in each sample. The highest estimated titres of co-sequenced plasmids and bacteriophages were identified and are provided in Table 9. The parentage for a given extrachromosomal plasmid cannot be attributed with full confidence to a particular species but the overwhelming likelihood suggests a direct relationship of these elements with the Lactobacillus crispatus cells detected in each sample. The method illustrated in Example 1 therefore allows a detailed in vivo picture to be determined of bacterial colony characteristics beyond the bacterial genome. The diversity of co-sequenced plasmids and their relative ratios with the estimated Lactobacillus crispatus levels provided a ‘fingerprint’ to rapidly differentiate different colonies of the same species. This allows the described method to monitor treatment efficacy or temporal infection studies in vivo by discriminating between re-emergence of a previous colony vs newly acquired infection from a different colony of the same species. Tracking of specific plasmids known to confer altered behavioural host characteristics such as acquired antimicrobial resistance can support epidemiological studies and aid appropriate medical interventions.

TABLE 9
Comparison of plasmid and phage cohorts in four different donor
samples with predominant Lactobacillus crispatus signals.

| | Urine samples | | | |
|---|---|---|---|---|
| | CP004_56 | CP008_71 | CP023_07 | CP0031_02 |
| Bacteria | (cells/mL) | (cells/mL) | (cells/mL) | (cells/mL) |
| Lactobacillus crispatus | 84,281,275 | 22,659,302 | 36,913,317 | 78,056,241 |
| Lactobacillus kefiranofaciens | 266,065 | 391,630 | 152,890 | 343,733 |
| Lactobacillus amylovorus | 877,395 | 395,353 | 443,297 | 759,869 |
| Lactobacillus acidophilus | 376,697 | 143,684 | 113,710 | 279,549 |
| Lactobacillus iners | None detected | None detected | None detected | 2,449,256 |
| Plasmids | (copies/mL) | (copies/mL) | (copies/mL) | (copies/mL) |
| Lactobacillus pLcAB70 | 321,534,040 | 101,324,713 | 179,467,780 | 284,578,591 |
| Lactobacillus FDAARGOS 743 | 87,640,936 | 2,564,034 | 41,434,196 | 71,784,645 |
| Lactobacillus DSM20075 pLH1 | 54,040,359 | None detected | None detected | None detected |
| Lactobacillus GRL1118.2 | 15,616,915 | 14,996,720 | 6,262,900 | 11,919,640 |
| Lactobacillus GRL1112.1 | 12,221,125 | 1,954,904 | 613,733 | None detected |
| Lactobacillus JV-V03.1 | 1,811,053 | 911,945 | 1,570,701 | 3,739,469 |
| Lactobacillus pFAM8627 | 815,834 | None detected | None detected | None detected |
| Lactobacillus pRMRA301 | 697,176 | 207,565 | 463,081 | 1,067,217 |
| Lactobacillus LJBSp1 | None detected | 4,319,866 | None detected | None detected |
| Lactobacillus pL6-1 | None detected | 896,164 | None detected | None detected |
| Phages | (copies/mL) | (copies/mL) | (copies/mL) | (copies/mL) |
| Lactobacillus AQ113 | 8,789,599 | 2,042,549 | 2,271,965 | 3,905,274 |
| Lactobacillus prophage Lj771 | 2,274,520 | 715,500 | 592,275 | 1,476,910 |

### Example 9—Analysis of Healthy Microbiome from Female Urine Samples

### Input Preparation

Samples were collected by twenty three asymptomatic healthy female volunteer donors using a provided home sampling kit containing 30 mL universal sodium borate urine tubes (Sterilab) and sterile hard-packed vaginal swabs (Scientific Laboratory Supplies). To minimise sample contamination donors were requested to clean around the urethra thoroughly using a sterile hygienic intimate wipe (Jeevson) before collecting their sample indirectly using a disposable sterile PeeCanter urine collection device (MedDX Solutions) All samples were received within 48 hours and stored at 4° C.

### Method of Example 1

DNA was extracted, sequenced and analysed according to the method of Example 1.

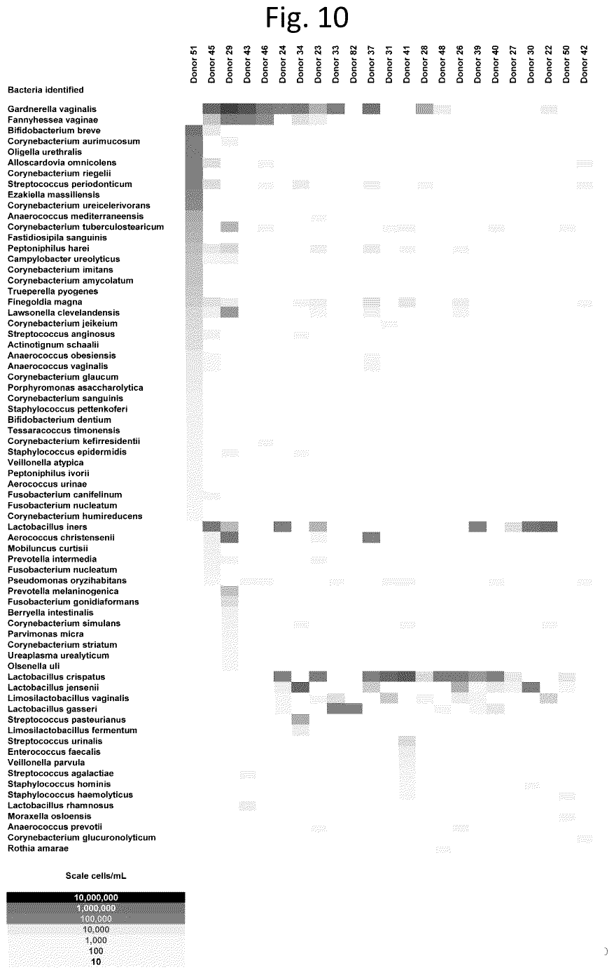

The method illustrated in Example 1 was used to profile biomes in urine samples taken from healthy female donors (FIG. 10). Initially, single time-point analysis of urine samples taken from 23 adult female volunteers (average age 31, median 24, range 18-53) was conducted. The total estimated bacterial load recovered for each urine sample varied considerably from 12,100 to 6,400,000 cells/mL with a median value of 590,000 cells/mL. This quantification of estimated bacterial load recovered for each urine sample represented 10× to 100× greater bacterial loads than those previously estimated using 16s rRNA methods (Pearce et al: The female urinary microbiome: a comparison of women with and without urgency urinary incontinence. mBio 2014, 5(4):e01283-0121). Between 1 and 37 discrete organisms were identified per sample, with the median value being 5 discrete organisms per sample. No obvious correlation existed between the number of organisms detected and the total cell titre measured. 70 different species were identified across the 23 sample cohort at levels >1,000 cells/mL. Gardnerella vaginalis (median 525,000 cells/mL) and Lactobacillus crispatus (median 253,000 cells/mL) were the most prevalent organism, each being present in 52% of the samples, followed by Lactobacillus jensenii (median 12,000 cells/mL) and Finegoldia magna (median 9,300 cells/mL), each in 39% of samples. 38 species were unique to single samples.

This analysis indicates discrete microbiome sub-types classified by the predominant species; Gardnerella vaginalis with Fannyhessia vaginae, Lactobacillus crispatus, Lactobacillus iners or Lactobacillus jensenii. Although a defined pattern exists for these ‘urobiome’ sub-types it also appears that a degree of overlap exists between them. No obviously common pattern was seen in the remaining samples.

Of interest, donor 51 provided a sample with the highest diversity microbiome listing 37 species dominated by high titres of Bifidobacterium breve at 1,130,000 cells/mL. 20/37 species identified are unique to this sample. Reports of rare involvement of Bifidobacterium species as agents of UTI suggest donor 51 may represent a potentially dysbiotic microbiome due to asymptomatic B. breve infection (Pathak P, Trilligan C, Rapose A: Bifidobacterium—friend or foe? A case of urinary tract infection with Bifidobacterium species. BMJ Case Rep 2014, 2014).

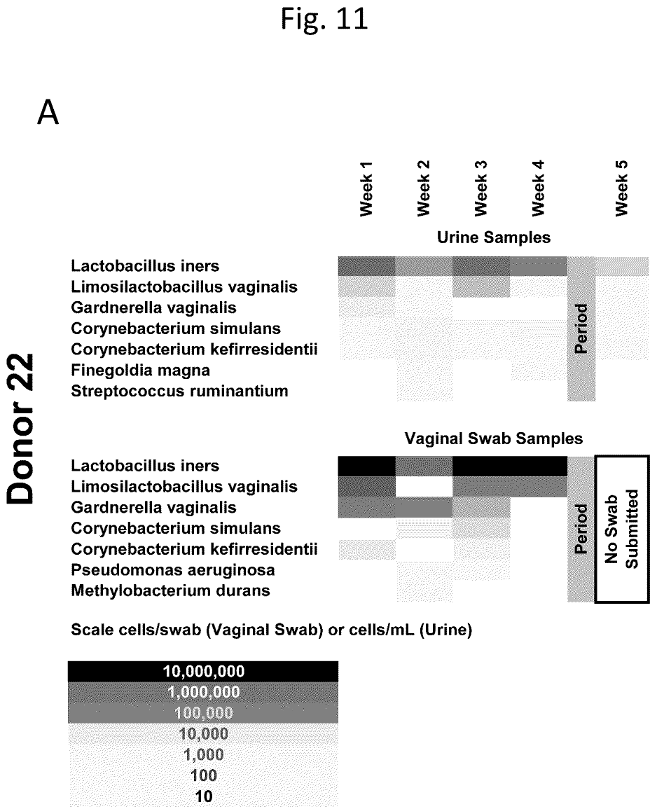

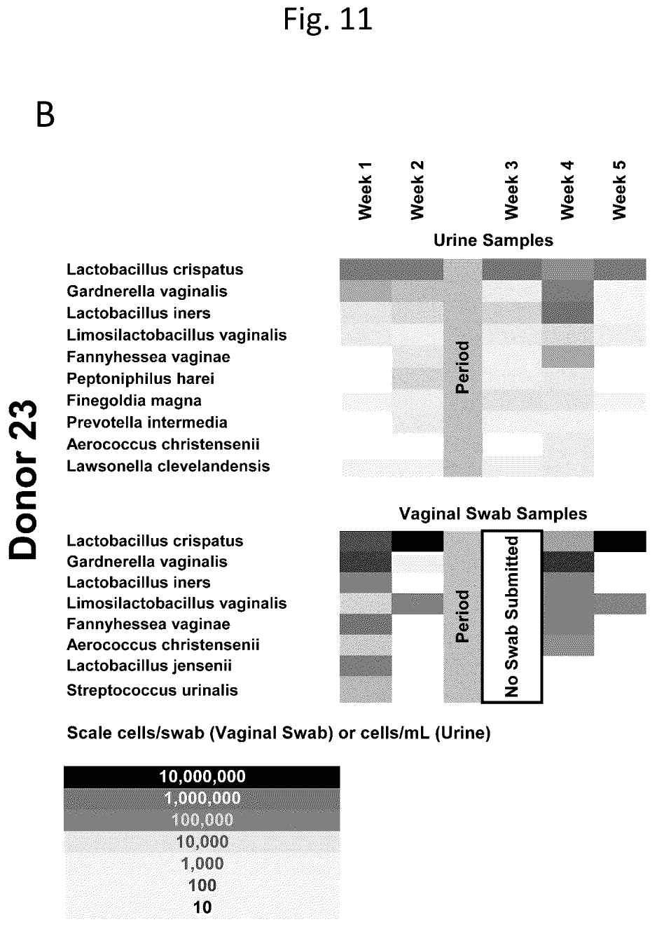

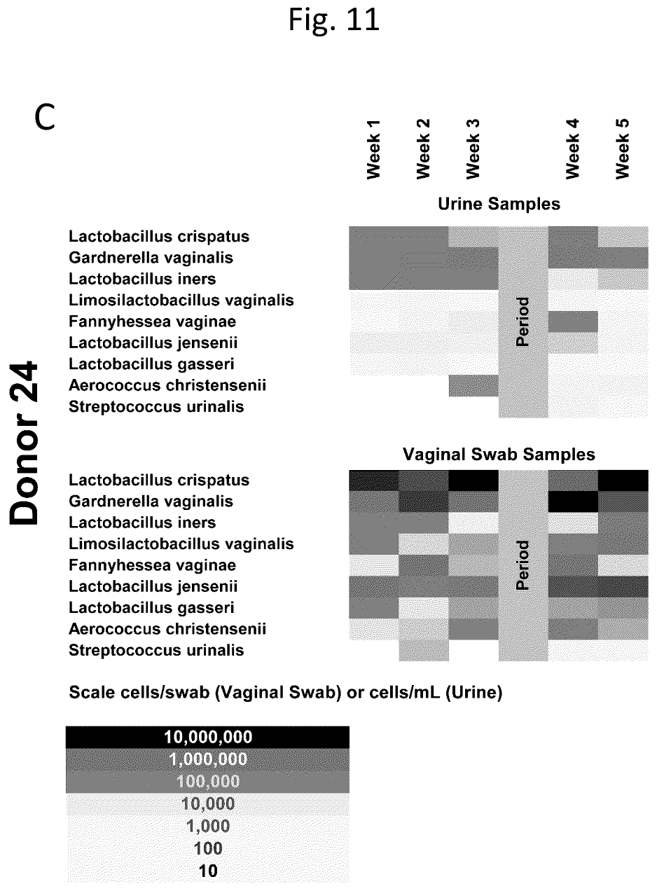

A more in-depth analysis was conducted on weekly samples from three volunteers to produce a temporal series spanning a full menstrual cycle (FIG. 11, top panels). Although largely reproducible, of interest was the dynamic changes observed in some female urinary biomes coincident with menstruation. Apparently stable microbiomes were seen to be transiently disrupted but rapidly re-established in the days or weeks that followed menstruation. These findings provide more resolution to previous studies using cpn60-based analysis describing similar phenomena in vaginal microbiomes (Albert et al: A Study of the Vaginal Microbiome in Healthy Canadian Women Utilizing cpn60-Based Molecular Profiling Reveals Distinct Gardnerella Subgroup Community State Types. PLOS One 2015, 10(8):e0135620, Chaban et al: Characterization of the vaginal microbiota of healthy Canadian women through the menstrual cycle. Microbiome 2014, 2:23).

### Example 10—Analysis of Healthy Microbiome from Female Vaginal Swab Samples

### Input Preparation

Samples were collected by nineteen asymptomatic healthy female volunteer donors using a provided home sampling kit containing 30 mL universal sodium borate urine tubes (Sterilab) and sterile hard-packed vaginal swabs (Scientific Laboratory Supplies). To minimise sample contamination donors were requested to clean around the urethra thoroughly using a sterile hygienic intimate wipe (Jeevson) before collecting their sample indirectly using a disposable sterile PeeCanter urine collection device (MedDX Solutions) All samples were received within 48 hours and stored at 4° C.

### Method of Example 1

DNA was extracted, sequenced and analysed according to the method of Example 1.

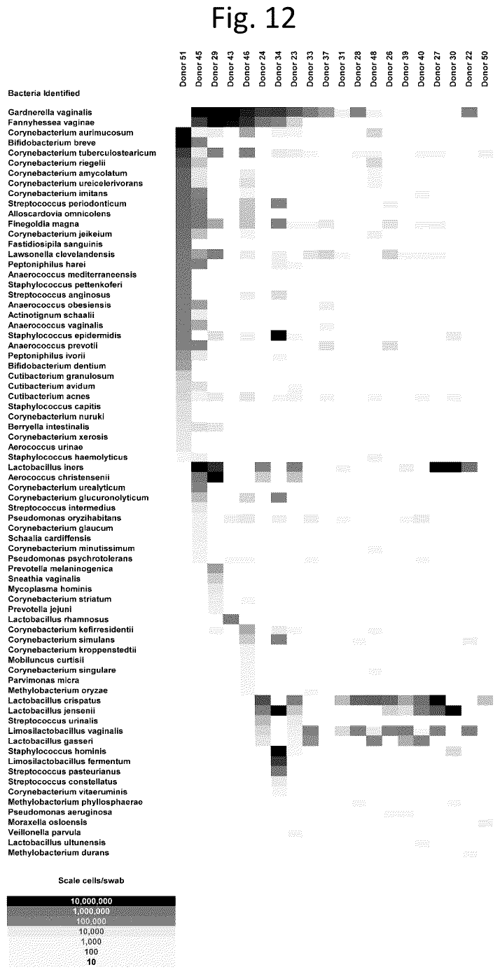

Vaginal swabs were also taken in parallel to each urinary test for 19 healthy donors enabling us to co-profile healthy vaginal biota (FIG. 12). Estimated cell values for each species identified at >1,000 bacterial cells/swab are given for each sample (FIG. 11) and allow estimates of the total number of cells recovered per swab (range 56,000 to 170,000,000 cells/swab, median 3,600,000 cells/swab). On average vaginal swabs return 5× greater titres of bacterial cells than an average titre/mL seen in urine samples from the same donor. As with the urine samples, the number of bacterial cells recovered for each vaginal swab varied considerably, some of this likely due to variation in individual donor swabbing technique. On average 14 discrete organisms (range 3-46, median 9) were identified per sample. A total of 87 different species were identified across the 19 sample cohort, 50% of which were also observed in the urine samples. As in the urine samples, Gardnerella vaginalis (average 22,510,000 cells/mL) was the most commonly observed organism, found in 65% of the vaginal swab samples, followed by Corynebacterium tuberculostearicum and Lawsonella clevelandensis, found in 60% of samples, then Limosilactobacillus vaginalis and Finegoldia magna in 55%. Forty-two species were unique to single donor samples.

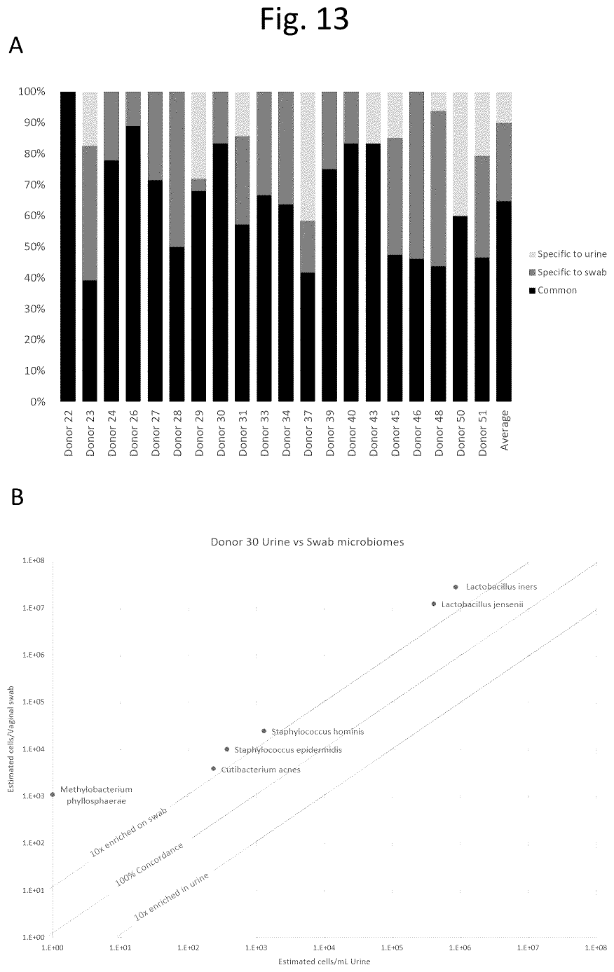

Evidence of discrete vaginal microbiomes characterised by the same dominant species identified in the urine analysis; Gardnerella vaginalis, Lactobacillus iners, Lactobacillus gasseri or Lactobacillus crispatus suggest a considerable overlap between the two populations measured from the same individual and agree with previous observations using cpn60 profiling as shown in FIGS. 10 & 12 (Chaban et al: Characterization of the vaginal microbiota of healthy Canadian women through the menstrual cycle. Microbiome 2014, 2:23). Correlation analysis between samples collected from the same donor on the same day suggests that urinary and vaginal microbiomes share an average 65% of identified species per individual (FIG. 13a). In addition, for most donors, the ratio of relative titres of bacterial species in their urinary and vaginal samples correlate, suggesting a tight relationship between these microbial communities (FIG. 13b). Species unique to urine samples represent a diverse mix of 14 different genera whereas, interestingly, nearly all species specific to the vaginal swab samples were from the genus Corynebacterium; C. urealyticum, C. imitans, C. riegeli, C. ureicelerivorans and C. amycolatum (FIGS. 10, 12 & 14).

Analysis of weekly vaginal swab samples taken during our temporal series (FIG. 11, bottom panels) shows that the urinary and vaginal microbiome from the same individual overlap considerably. Where menstrual shedding was seen to result in a transitory change in urinary microbiome dominance, coincident change was also observed in the vaginal swab. Of interest however, is that the nature of these transient changes seem specific to each location; Lactobacillus crispatus and Lactobacillus iners urine ratios become transiently inverted in donor 23 & 24 samples whilst Gardnerella vaginalis and Lactobacillus crispatus ratios display a similar phenomenon in the vagina in the same donors over the same time frame.

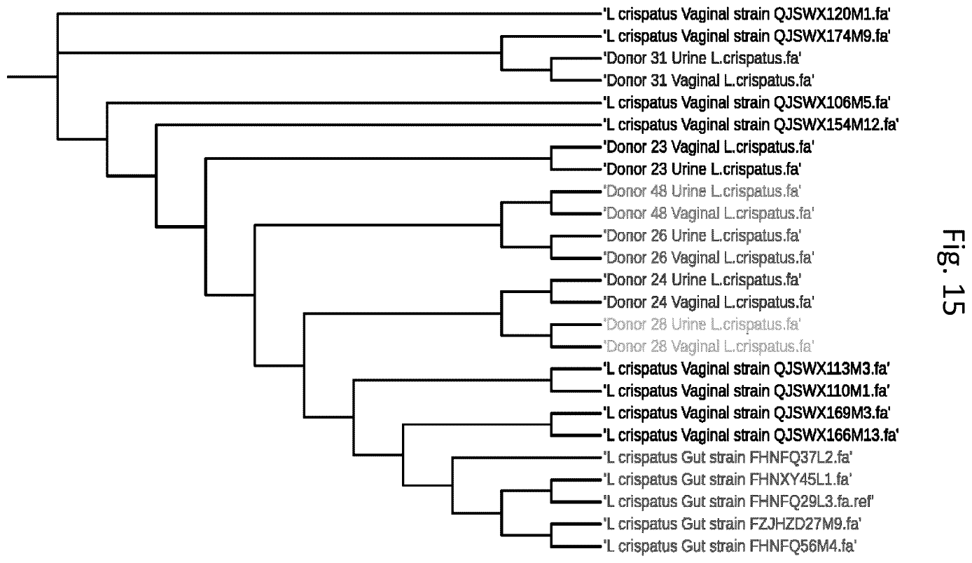

The method illustrated in Example 1 is able to compute consensus genome references for all bacterial species present in a sample with greater than ten-fold aligned sequence coverage. Extracted references can be compared using genome-wide SNP comparison tools to produce cladograms highlighting relationships to key published reference strains of the same species. Six of our healthy control donors displayed Lactobacillus crispatus levels with sufficient coverage to construct independent consensus references for both their urine and vaginal swab samples. Comparison of these references against previously published vaginal and gut strains (Zhang et al: Comparative Genomics of Lactobacillus crispatus from the Gut and Vagina Reveals Genetic Diversity and Lifestyle Adaptation. Genes (Basel) 2020, 11(4)). shows that, in all six cases, the closest homologies were between urine and vaginal L. crispatus references computed from the same donor (FIG. 15). This pairing of references strongly suggests that urine and vaginal strains are shared in the same individual but also that these strains are unique to donors. Of note, all consensus references show highest homology to published vaginal rather than gut strains, further supporting the hypothesised anatomical niche-specific strain adaptation of this genetically diverse species (Zhang et al: Comparative Genomics of Lactobacillus crispatus from the Gut and Vagina Reveals Genetic Diversity and Lifestyle Adaptation. Genes (Basel) 2020, 11(4)).

### Example 11—Analysis of Urine Sample Contamination

### Input Preparation

Samples were collected by ten asymptomatic healthy female volunteer donors using a provided home sampling kit containing 30 mL universal sodium borate urine tubes (Sterilab) and sterile hard-packed vaginal swabs (Scientific Laboratory Supplies). To minimise sample contamination donors were requested to clean around the urethra thoroughly using a sterile hygienic intimate wipe (Jeevson) before collecting their sample indirectly using a disposable sterile PeeCanter urine collection device (MedDX Solutions) All samples were received within 48 hours and stored at 4° C.

### Method of Example 1

DNA was extracted, sequenced and analysed according to the method of Example 1.

Urine collection methods can be prone to contamination by, for example, co-capturing local epithelial microbial populations during sample collection. To reduce these concerns, we included the use of a single-use intimate hygienic wipe for donors to clean around the urethral entrance prior to urine collection. To assess the efficacy of using a wipe to reduce contaminants further analysis was conducted on a separate cohort of ten female donors who were asked to use sterile swabs to capture peri-urethral epithelial samples from the vulva before and after using the wipe. Following wipe donors were asked to capture urine and vaginal swab samples for microbiome profiling. A correlation analysis between the urine sample and vaginal swab samples microbial populations was conducted to establish the impact of surface contamination in the method.

A total of 52 species were detected at >1,000 cells on peri-urethral epithelial swabs collected from ten female donors before they used a wipe. A subset of seven species; C. glucuronolyticum, L. iners, F. magna, P. harei, A. obesiensis, C. tuberculostearicum, S. periodonticum, were found in over half the samples tested.

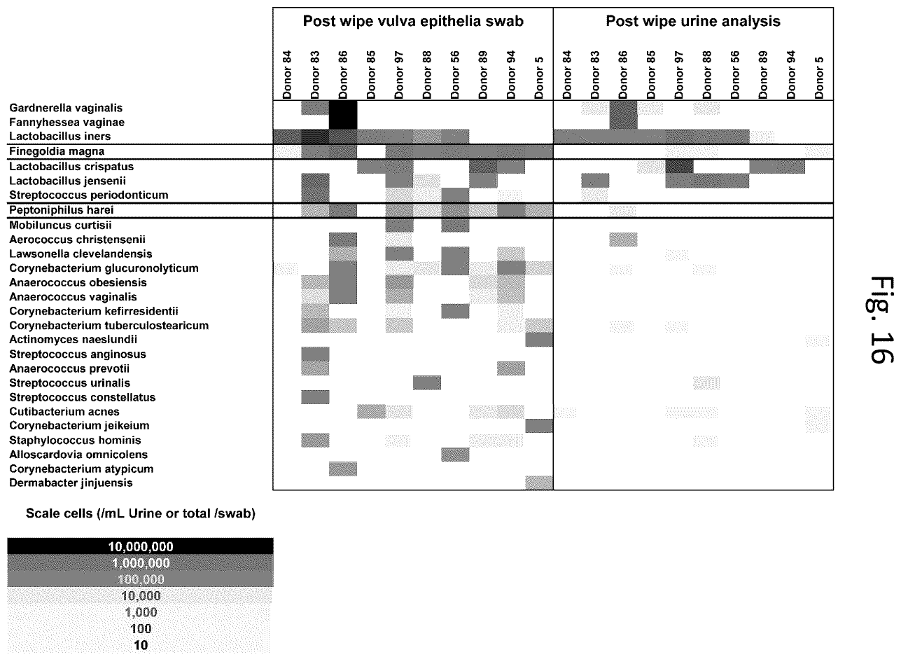

Comparison of paired swabs taken from the same individual before and after using the wipe showed that, where the same species was still present after wiping, relative cell titres had been reduced by an average of 4.9× (median 2.7×) indicating that for most species this is a practical method of reducing epithelial sample contamination. High degrees of concordance exists between the microbial profiles of urinary, vaginal and vulva epithelial sample sets collected from the ten female donors which might be expected due to physical proximity of these sites. However, the impact of putative epithelial microbiome contamination on urinary samples can still be assessed using key indicator species; organisms that are heavily enriched in epithelial swab samples yet depleted or entirely absent from urine samples from the same donor. Across the ten female datasets collected, two species, Finegoldia magna and Peptoniphilus harei, conformed to this profile, being highly prevalent in epithelial samples, but either individually absent or both absent from urinary microbiomes of the same donor. We observed this pattern in 8/10 of the individuals tested (FIG. 16). This suggests that contamination by these species, and likely therefore other organisms associated with the microbiome detected on vulva epithelium, is not a significant factor in urinary microbiome profiling with our method.

### Example 12—Analysis of Healthy Microbiome from Male Urine Samples

### Input Preparation

Samples were collected by eighteen asymptomatic healthy male volunteer donors using a provided home sampling kit containing 30 mL universal sodium borate urine tubes (Sterilab) and sterile hard-packed vaginal swabs (Scientific Laboratory Supplies). To minimise sample contamination donors were requested to clean around the urethra thoroughly using a sterile hygienic intimate wipe (Jeevson) before collecting their sample indirectly using a disposable sterile PeeCanter urine collection device (MedDX Solutions) All samples were received within 48 hours and stored at 4° C.

### Method of Example 1

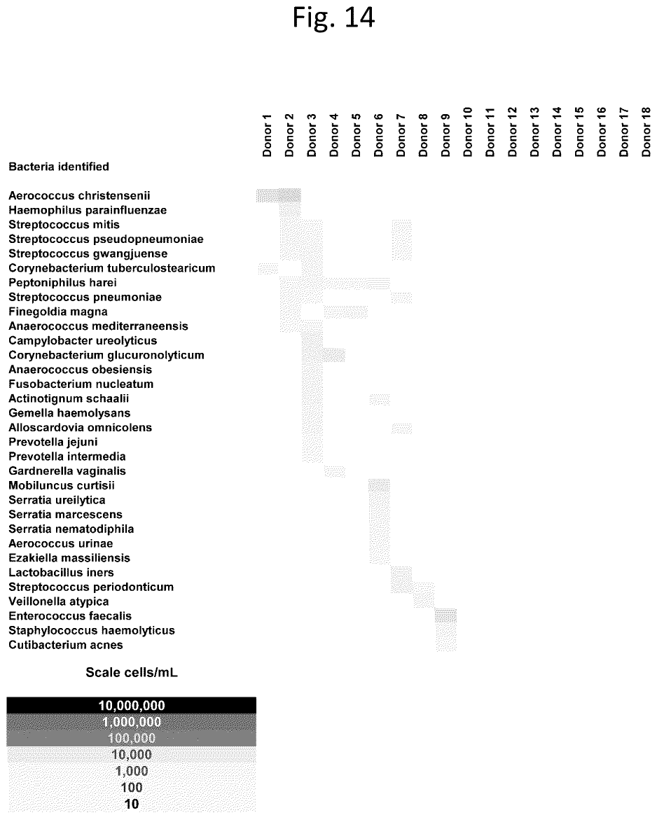

DNA was extracted, sequenced and analysed according to the method of Example 1. The sex difference of urinary microbiomes in healthy individuals was investigated. Urine samples from 18 healthy male volunteers (average age 43, median 40, range 19-72, FIG. 14). Lower numbers of species were observed in each sample with 9/18 failing to return any organisms present at >1,000 cells/mL. On average, the 9 male urine samples with measurable organisms returned ˜200× lower average total estimated cell/mL values when compared to female urine samples (range 4,500 to 49,000 cell/mL, median=20,000 cells/mL). Samples from this group had between 2 and 16 (average 3) discrete organisms, the most prevalent of which, Peptoniphilus harei, was present in 5/17 samples. Of note, if thresholds for reporting were reduced to lower than 1,000 cells/species, bacteria characteristic for the ‘kit-ome’, Cutibacterium acnes, Pseudomonas stutzeri and Pseudomonas aeruginosa were seen to be present across all 18 samples suggesting that these samples approached the lowest practical measurable limit of the method.

Previous reports suggest enrichment of Corynebacterium and Streptococcus species in male urine [15, 24]. We confirm the presence of five Streptococcus species present in more than one sample; S. gwangjuense, S. mitis, S. periodonticum, S. pneumonia and S. pseudopneumoniae as well as two species of Corynebacterium; C. glucuronolyticum and C. tuberculostearicum (FIG. 14).

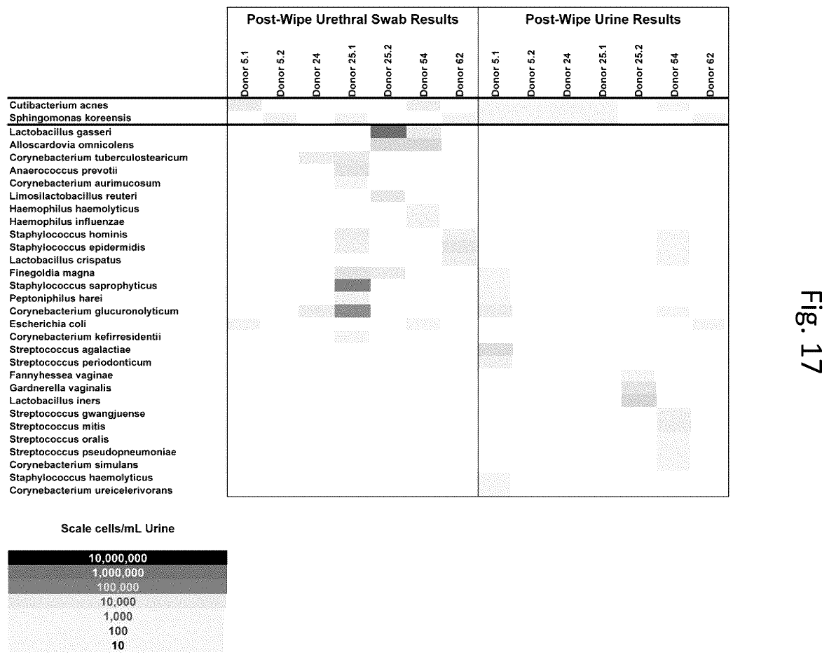

Akin to our previous study on female samples, we investigated the contribution of local epithelial microbiome contamination on samples by analysing peri-urethral epithelium swabs collected by a new cohort of seven healthy male donors after using a sterile intimate cleansing wipe across the respective area. Results were compared to the urine sample profiles collected immediately after cleaning and swabbing (FIG. 17). Once low level contributions from ‘kit-ome’ species Sphingomonas koreensis and Cutibacterium acnes were excluded we found very little commonality between the composition of epithelial and urinary microbiomes of the same individual, despite several displaying moderate biomasses in their epithelial swabs. This suggests that, where urinary organisms were detected, they had not arisen through epithelial contamination during sample collection. Similar to our previous data, 3/7 of the samples appeared practically sterile.

Comparison of urinary microbiomes between healthy control males and females showed that 27/60 species identified are common to both and, surprisingly, were present at similar average titres+/−10×. Given the marked difference in total estimated bacterial biomass between male and female urine samples this underlines the majority contribution made by species that are unique to or heavily enriched in the female urine samples such as Gardnerella vaginalis, Lactobacillus iners and Lawsonella clevelandensis. A total of 20/69 organisms detected were restricted to female samples including Fanyhessea vaginae and six species of the genus Lactobacillus/Limosilactobacillus; L. crispatus, L. jensenii, L. gasseri, L. rhamnosus, L. fermentum and L. vaginalis. In comparison, Lactobacillus species were almost completely absent in male samples. 13/60 species were specific to male samples including four of the Streptococcus genus; S. gwangjuense, S. mitis, S. pneumonia and S. pseudopneumoniae and three species of the genus Serratia, S. urielytica, S. marcescens and S. nematodiphila, all identified at relatively low titres.

### Discussion

The above examples demonstrate that the method described herein, as illustrated by Example 1, is capable of accurately co-identifying both relative and absolute quantities of multiple gram-negative, gram-positive and yeast species with high reproducibility and very little bioinformatic noise. In addition, the method has the sensitivity to quantitatively detect individual bacterial species at or above 1×10³ cells total input, some 100× more sensitive than the 1×10⁵ cut-off currently advised by NHS culture-based reporting guidelines (PHE: SMI B41: investigation of urine. Information on UK standards for microbiology investigations of urine. Public Health England 2018). The method was also used to re-investigate the composition, dynamics and interplay between healthy female urine/vaginal and male urinary microbiomes. Design and interpretation of such analyses can be complex as organisms inhabiting the epithelium of the vulva, urethra or even reagents and receptacles used to collect and process samples have the capacity to contribute to final datasets. There is active debate in UTI research as to which of these populations are relevant, which are likely co-captured by different urine sample collection methods (Wolfe et al: Evidence of uncultivated bacteria in the adult female bladder. J Clin Microbiol 2012, 50(4):1376-1383), how sample storage and transport impacts microbiome fidelity (Daley, Gill, Midodzi: Comparison of clinical performance of commercial urine growth stabilization products. Diagn Microbiol Infect Dis 2018, 92(3):179-182) and how, ultimately, variability can be controlled in clinical application.

The experiments used a highly practical voided ‘first-catch’ donor self-collection protocol using a dedicated single-use disposable collecting device decanted into a Sodium Borate (Daley, Gill, Midodzi: Comparison of clinical performance of commercial urine growth stabilization products. Diagn Microbiol Infect Dis 2018, 92(3):179-182) containing tube for transport. Investigations were conducted into the contribution of each potential source of variability in this system. A reproducible ‘kit-ome’ of low titre organisms introduced during processing was defined and informs future thresholding and filtering of these species. The composition of the vulval microbiome around the urethra has also been defined and shown that the use of a sterile single-use intimate wipe before sample collection reduces the local biomass significantly. Key high-prevalence peri-urethral microbiome indicator species such as Finegoldia magna and Peptoniphilus harei were also identified as not present in urine samples of the same female donors and demonstrate that little overlap exists in urine and epithelial microbiomes in males. Together this suggests minimal urine sample cross-contamination from these sources.

Sample variability can be compounded further by choice of analytical technique. For example, methods such as standard culture are subject to heavy selection bias for fast-growing aerobic species whereas PCR-based assays including 16s rRNA profiling have high innate sensitivity and are prone to report false-positives in low biomass samples (Kennedy et al: Questioning the fetal microbiome illustrates pitfalls of low-biomass microbial studies. Nature 2023, 613(7945):639-649). In contrast, the method described herein, as illustrated in Example 1, is able to be completely PCR and culture free and to take raw urine directly as input to minimise biasing issues whilst maintaining high sensitivity.

The results confirm previous, often qualitative, findings that describe commonality between urinary and vaginal microbial populations. Until now this relationship has been largely based on 16s rRNA analyses of operational taxonomic units (OTUs) that give relative or ratio-metric rather than absolute abundances and are often restricted to family or genus level observations (Wolfe et al: Evidence of uncultivated bacteria in the adult female bladder. J Clin Microbiol 2012, 50(4):1376-1383, Pearce et al: The female urinary microbiome: a comparison of women with and without urgency urinary incontinence. mBio 2014, 5(4):e01283-01214, Gottschick et al: The urinary microbiota of men and women and its changes in women during bacterial vaginosis and antibiotic treatment. Microbiome 2017, 5(1):99, Pohl et al: The Urine Microbiome of Healthy Men and Women Differs by Urine Collection Method. Int Neurourol J 2020, 24(1):41-51., 27, 28). The above examples provide important and much needed extra resolution by moving to reproducible and robust quantitative species level identification. We confirm previous reports that the Lactobacillus genus is highly enriched in female urine (Fouts et al: Integrated next-generation sequencing of 16S rDNA and metaproteomics differentiate the healthy urine microbiome from asymptomatic bacteriuria in neuropathic bladder associated with spinal cord injury. J Transl Med 2012, 10:174, Modena et al: Changes in Urinary Microbiome Populations Correlate in Kidney Transplants With Interstitial Fibrosis and Tubular Atrophy Documented in Early Surveillance Biopsies. Am J Transplant 2017, 17(3):712-723) and vaginal samples but also show a complex interplay between several Lactobacillus species. Further, these relationships appear dynamic across a menstrual cycle suggesting that the change to local physical and endocrine conditions have transient but significant effects on microbiome compositions in vivo. We confirm that Gardnerella and Lactobacilli dominate the majority of urobiome types in healthy females (Gottschick et al: The urinary microbiota of men and women and its changes in women during bacterial vaginosis and antibiotic treatment. Microbiome 2017, 5(1):99) but can extend these observations to provide evidence of at least three sub-urobiome types dominated by individual Lactobacillus species. The results confirm the largely exclusive relationship previously observed between L. crispatus and G. vaginitis (Castro et al: Reciprocal interference between Lactobacillus spp, and Gardnerella vaginalis on initial adherence to epithelial cells. Int J Med Sci 2013, 10(9):1193-1198, Ojala et al: Comparative genomics of Lactobacillus crispatus suggests novel mechanisms for the competitive exclusion of Gardnerella vaginalis. BMC Genomics 2014, 15:1070) and show that this relationship generally holds true in vivo for both urinary and vaginal microbiomes.

In comparison male urobiomes have two orders of magnitude lower biomass on average and confirm previous observations that they are enriched for species of the Corynebacterium and Streptococcus genera but are now able to provide species-level identifications and absolute quantitation. Of interest, half of the input healthy male urine samples returned no organisms other than those compatible with contribution from the process kit-ome. Hence, they may be considered practically sterile as assayed by our technique. The high frequency of this finding in the male samples raises the possibility that, in stark contrast to females, this may represent the normal status of healthy male urine.

Analysis of Lactobacillus crispatus donor-specific strains using genome consensus mappings computed directly from sample sequencing data show that dominant strains have donor-specific SNP patterns. Further, these patterns are most closely shared by vaginal and urinary strains of the same individual underlining the tight relationship between microbiomes at these locations. Automatic differentiation of bacterial strains at genome wide SNP level directly from urine or swab samples will prove useful as both a research and clinical tool in looking at, for example, identifying pathogenic sub-strains of bacterial species or establishing re-infection versus relapse in recurrent E. coli infections of the same individual.

### CONCLUSION

The method described herein provides a cost-effective, rapid, unbiased and fully-quantitative microbiome profiling tool.

It has been embedded into a semi-automated end-to-end workflow enabling home kit sampling, sample tracking, processing, data analysis and results reporting, usually within 48 hrs of sample capture.

This has provided a fit-for-purpose clinical UTI diagnostic tool for cases where patients have; classic UTI symptoms caused by undetected infection; non-classical UTI symptoms misdiagnosed as alternative conditions; or cyclical relapse resulting from failure to eradicate a previously identified pathogen.

The described workflow can be applied directly to help the numerous women debilitated by chronic or recurrent UTIs that are served poorly by the current diagnostic systems.

The method, as illustrated above was able to produce fully quantitated clinically appropriate profiles for organisms present at >1,000 cells/mL from approximately 100,000 >1 kbp reads captured overnight. This same method can be readily modulated to allow different levels of sensitivity. Clinical applications requiring rapid and reliable identification of high-titre species, e.g. sepsis or pneumonia, can plausibly be made by monitoring a live data stream within minutes of sequence run initiation (Baldan et al: Development and evaluation of a nanopore 16S rRNA gene sequencing service for same day targeted treatment of bacterial respiratory infection in the intensive care unit. J Infect 2021, 83(2):167-174, Sheka et al: Oxford nanopore sequencing in clinical microbiology and infection diagnostics. Brief Bioinform 2021, 22(5)). Accurate profiling of low biomass microbiomes or highly dilute substrates for research applications may require the use of larger format flow cells that yield millions of reads when run over several days.

The application of molecular techniques is rapidly changing the landscape of urogenital microbiome profiling and infection diagnostics. Appropriate guidance for interpretation and clinical application of such data is, however, currently lagging. Development of such advice is pressing and has the potential to prevent the immediate clinical application of these important tools. The creation of appropriate guidance is reliant on suitably powered research studies to define healthy and dysbiotic microbiomes in the relevant anatomical systems. The data presented here should be useful in helping shape appropriately scaled research projects to meet this purpose.

1.-24. (canceled)

25. A method for detecting and quantifying the presence of an organism in a sample, wherein the method comprises:
determining an amount of sequenced organism nucleic acid sequence in the sample using sequencing data comprising at least one sequenced organism nucleic acid sequence obtained from sequencing organism nucleic acid from the sample; and
quantifying an amount of the organism based on the amount of the sequenced organism nucleic acid sequence.

26. The method of claim 25, wherein
the sequencing data further comprises one or more nucleic acid sequences sequenced from one or more reference organisms in the sample; and
quantifying the amount of the organism based on the amount of the sequenced organism nucleic acid sequence comprises comparing the amount of the sequenced organism nucleic acid sequence to an amount of the one or more nucleic acid sequences sequenced from the one or more reference organisms, optionally wherein the total amount of the organism nucleic acid is used to calculate the cell number of the organism in the sample.

27. The method of claim 26, wherein:
quantifying the amount of the organism further comprises determining a recovery ratio;
the recovery ratio is a ratio of the amount of the nucleic acid sequence sequenced from the one or more reference organisms to an expected amount of nucleic acid in the sample from the one or more reference organisms; and
the expected amount is based on an amount of the reference organism added to the sample prior to sequencing and, optionally, on a genome length of the one or more reference organisms, optionally wherein quantifying the amount of the organism comprises:
estimating a total amount of the organism nucleic acid using the amount of the organism nucleic acid sequence and the recovery ratio; and
estimating the amount of the organism in the sample based on the total amount of the organism nucleic acid and a genome length of the organism.

28. The method of claim 25, wherein the method comprises identifying the sequenced nucleic acid sequence as specific to a particular organism, optionally by comparison with a database of example organism nucleic acid sequences.

29. The method of claim 28, wherein identifying the sequenced nucleic acid sequence as specific to a particular organism comprises sequence alignment with one or more of the example organism nucleic acid sequences, optionally wherein the sequence alignment comprises alignment of the sequenced nucleic acid sequence to a reference organism nucleic acid sequence over its entire length, optionally by BLAST.

30. The method of claim 29, wherein:
the one or more example organism nucleic acid sequences comprises a plurality of example organism nucleic acid sequences; and
identifying the sequenced nucleic acid sequence as specific to the organism further comprises determining a most likely example organism nucleic acid sequence based on one or more relative mapping metrics.

31. The method of claim 30, wherein
a) the relative mapping metrics include: level of sequence identity; homology and/or length of match; and specific insertions, deletions and/or single nucleotide polymorphisms with respect to the example organism nucleic acid sequences;
b) determining the most likely example organism nucleic acid sequence comprises, in a case where the homology of the sequenced nucleic acid sequence with the example organism nucleic acid sequences is similar for a plurality of the example organism nucleic acid sequences, determining the most likely example organism nucleic acid sequence based on ratios of the plurality of the example organism nucleic acid sequences determined as the most likely example organism nucleic acid sequence from sequence alignment of others of the sequenced nucleic acid sequences; and/or
c) the method further comprises identifying position-specific sequence differences between the sequenced nucleic acid sequences and the corresponding most likely example organism nucleic acid sequence, optionally wherein the position-specific sequence differences comprise at least one of sequence polymorphisms, insertions, and deletions.

32. The method of claim 31, wherein:
(a) the identified position-specific sequence differences are weighted using error data representing a likelihood of sequencing errors in the sequencing of the at least one sequenced organism nucleic acid sequence; or
(b) wherein the method further comprises calculating a frequency measure for one or more of the position-specific sequence differences representing the frequency of the position-specific sequence differences across plural of the sequenced organism nucleic acid sequences in the sequencing data, optionally further comprising calculating a probability of the presence of a plurality of highly homologous organisms based on the frequency measures, optionally further comprising calculating a relative ratio between the highly homologous organisms.

33. The method of claim 25, wherein the sample is obtained from a host, the organism is a non-host organism and any reference organism is a non-host organism, further wherein optionally:
a) the host is a mammal, optionally wherein the host is a human; and/or
b) the non-host organism is a micro-organism and/or a parasite, optionally wherein the microorganism is a bacterium, a virus, a parasite, a bacteriophage, or a fungus.

34. The method of claim 33, wherein the non-host organism is a pathogen.

35. The method of claim 34, wherein
a) the detection and quantification of the non-host organism identifies a pathogen most likely responsible for an infection or disease in the host, optionally wherein the disease is a systemic infection, a local infection, a urinary tract infection, an infection of the blood, digestive tract infection, a central nervous system infection, a cardiovascular infection, an intro-abdominal infection, a urogenital tract infection, a genital tract (such as vaginal) infection, a respiratory infection and/or a skin infection; and/or
b) the method further comprises selecting an agent suitable to treat the pathogen, optionally wherein the agent is an antibiotic or an antifungal.

36. The method of claim 25:
(A) wherein the at least one sequenced organism nucleic acid sequence is greater than 500 bp, 750 bp, or 1000 bp in length, optionally wherein:
a) the sequenced organism nucleic acid sequence is the whole genome sequence of the organism; or
b) multiple organism nucleic acid sequences are sequenced to provide the whole genome sequence of the organism; or
(B) wherein
a) the method further comprises identifying one or more antimicrobial resistance genes in the sequenced organism nucleic acid sequences, optionally by sequence alignment of the sequenced organism nucleic acid sequences to one or more example antimicrobial resistance gene sequences;
b) the method further comprises identifying one or more plasmids and/or phages in the sample based on the sequencing data, optionally by comparison of the sequenced organism nucleic acid sequences with one or more example plasmid and/or phage nucleic acid sequences; and/or
c) the sample is obtained from a host, the organism is a non-host organism and the method further comprises estimating a probability of relapse and/or reinfection of the host by the non-host organism based on the amount of the non-host organism.

37. A method for detecting and quantifying the presence of an organism in a sample, wherein the method comprises:
obtaining organism nucleic acid from the sample;
sequencing at least one organism nucleic acid sequence using the organism nucleic acid to obtain sequencing data; and
detecting and quantifying the presence of the organism using the method of claim 25.

38. The method of claim 37, wherein:
a) the detection and quantification does not require amplification of the organism nucleic acid sequence;
b) the sample is obtained from a host, the organism is a non-host organism and the method comprises substantial depletion of host cells from the sample obtained from the host;
c) the method comprises the addition of a known quantity of at least one reference organism to the sample;
d) the organism is cellular and the method comprises substantial lysis of the organism cells, optionally wherein the lysis of organism cells is performed by enzymatic digestion, thermal and/or physical disruption and optionally the lysis of non-host organism cells is performed by enzymatic digestion, optionally using proteinase K, bead bashing, thermal disruption and/or sonication;
e) the method comprises generating a sequencing library from the organism nucleic acid;
and/or
f) the sequencing is nanopore sequencing.

39. The method of claim 37, wherein:
(A) the sample is obtained from a host, the organism is a non-host organism and:
(i) detection and quantification of the non-host organism does not require amplification of the non-host nucleic acid sequence;
(ii) the method comprises substantial depletion of host cells from the sample;
(iii) the method comprises addition of a known quantity of at least one reference non-host organism to the sample;
(iv) the method comprises substantial lysis of non-host organism cells;
(v) the method comprises generating a sequencing library from the non-host nucleic acid;
(vi) the method comprises sequencing at least one non-host organism nucleic acid sequence of greater than 500 bp, 750 bp, or 1000 bp in length; and
(vii) the method comprises identifying the sequenced non-host nucleic acid sequence as specific to a particular non-host organism by alignment of the sequenced non-host nucleic acid sequence to an example non-host organism nucleic acid sequence over its entire length, optionally by BLAST; or
(B) the sample is obtained from a host, the organism is a non-host organism and:
a) the non-host nucleic acid is not extracted or purified from the sample prior to sequencing;
b) the-non-host organism is a bacterium and the method comprises sequencing non-genomic DNA of the non-host organism, optionally wherein the non-genomic DNA is a plasmid and/or bacteriophage;
c) the method further comprises sequencing of one or more antimicrobial resistance genes of the non-host organism;
d) the method is conducted on a saliva, blood, urine, tissue, mucus, vaginal swab, faeces, semen, spinal fluid and/or plasma sample obtained from the host; and/or
e) the method is conducted on a urine sample from the host and the non-host organism is a bacterium and wherein the detection and quantification of the bacterium identifies the bacterium as a pathogen most likely responsible for a urinary tract infection.

40. A method of treating a disease or infection associated with a pathogen in a host, wherein the method comprises detecting and quantifying the pathogen according to the method of claim 25 and administering an agent suitable to treat the pathogen, optionally wherein the agent is an antibiotic or an antifungal.

41. A method of monitoring the effectiveness of a treatment of a disease or infection associated with a pathogen in a host, wherein the method comprises detecting and quantifying the pathogen according to the method of claim 25 and determining whether the treatment decreases the quantity of the pathogen in a sample obtained from the host.

42. A kit comprising:
(i) a means for depleting cells from a sample;
(ii) one or more reference organisms in known quantities; and
(iii) a means for generating a sequence library from nucleic acids, optionally wherein the cells are host cells and the sample is obtained from a host sample, the reference organisms is are non-host organisms, and the nucleic acids are non-host nucleic acids.

43. An apparatus for detecting and quantifying the presence of an organism in a sample, the apparatus comprising:
a determining unit configured to determine an amount of sequenced organism nucleic acid sequence in the sample using sequencing data comprising at least one sequenced organism nucleic acid sequence obtained from sequencing organism nucleic acid from the sample; and
a quantifying unit configured to quantify an amount of the organism based on the amount of the sequenced non-host organism nucleic acid sequence, optionally wherein the sample is obtained from a host, the organism is a non-host organism and the reference organism is a non-host organism.

44. A computer program comprising instructions which, when the program is executed by a computer, cause the computer to carry out the method of claim 25.

45. A computer-readable storage medium comprising instructions which, when executed by a computer, cause the computer to carry out the method of claim 25.
